(19) United States
(12) Patent Application Publication
(10) Pub. No.: US 2013/0149740 A1
(43) Pub. Date: Jun. 13, 2013

(54) METHOD FOR THE PRODUCTION OF A POLYMERIZED PRODUCT

(71) Applicant: Yves Delmotte, Neufmaison (BE)

(72) Inventor: Yves Delmotte, Neufmaison (BE)

(73) Assignees: Baxter Healthcare S.A., Glattpark (Opfikon) (CH); Baxter International Inc., Deerfield, IL (US)

(21) Appl. No.: 13/523,328

(22) Filed: Jun. 14, 2012

Related U.S. Application Data
(60) Provisional application No. 61/496,725, filed on Jun. 14, 2011.

(51) Int. Cl. C12P 21/00; C07K 1/02

(52) U.S. Cl. CPC C12P 21/00; C07K 1/02
USPC 435/68.1; 422/131; 435/289.1; 530/356; 530/382

(57) ABSTRACT

The invention discloses a method for the production of a polymerized product comprising the following steps:
providing a polymerization device to which a polymerization mixture and a separation medium can be applied and wherein flow of said mixture and medium can be conducted in appropriate ducts for said mixture and medium,
transporting said polymerization mixture in a duct of said polymerization device thereby allowing the polymerization reaction,
transporting said mixture in a duct of said polymerization device in a continuous flow,
interrupting said continuous flow of said mixture with said separation medium so as to obtain consecutive volumes of said mixture and volumes of said separation medium,
further transporting said consecutive volumes of said mixture and volumes of said separation medium in a duct of said polymerization device wherein said mixture further polymerizes to obtain a discontinuous polymerized product, and
   removing said discontinuous polymerized product from said polymerization device.

### FIELD OF THE INVENTION

The present invention relates to the field of producing polymerized products, especially fibrin products for medical use.

### BACKGROUND OF THE INVENTION

Biologic absorbable implant material's for filling and closing soft-tissue cavities or bone cavities and for replacing soft- and bone-tissue parts, as well as to a method of its preparation are known in the art. Such materials can also be used in fields such as drug delivery surgeries and tissue engineering. Besides tissue filling the materials can also be used out of the body for filling, coating, covering devices (synthetic, biologic, etc.) that will be implanted such as spine fusion cages, grafts, etc.

In the field of orthopaedics, implant materials for filling bone cavities may e.g. be produced by partial deproteinization and denaturation of the residual protein from spongiosa bone tissue. Various materials, especially those based on natural components have been suggested, e.g. derived from collagen and fibrin or based on bone structures (e.g. decalcified tubular bones). For example, bone collagen can be decalcified and lyophilized and forms an osteoinductive gel upon reconstitution. Synthetic materials, such as acrylates, have likewise been proposed for such purposes as well as prosthetic implant materials produced from body tissues by treatment with protein cross-linking agents.

Such implant material has in general to be well-tolerated material that may be used to close tissue cavities, on the one hand, and to substitute certain tissue parts, on the other hand. In thorax surgery sealing and curing bronchial fistulas is also problematic, in particular, if the introduction of the implant is to be effected by way of endoscopy, which is the quickest and mildest way. Special demands are required of an implant material to be introduced and fixed endoscopically. On the one hand, mechanical spreading must be feasible; on the other hand, introduction through the bronchial tree to the fistula must be possible. Such implants must be deformable and compressible. It should be able to reassume its original shape in the presence of moisture, i.e., it must have at least some kind of memory effect. In addition, the material must offer a considerable flexibility, yet remain absorbable, because germs may adhere to non-absorbable materials, thus causing abscesses and new fistulas again and again. Such material should also allow filling of gaps and cavities of unknown or unexpected (at least not predeterminable) size, e.g. during surgery.

Fibrin or collagen blocks, beads or microbeads have been used in the past for fulfilling such needs; however, such beads are often problematic in handling because of their particulate nature.

There is a need for providing material of the described nature which may be specifically suited in surgery, especially for providing bioresorbable implants. It is an object to provide such material and appropriate methods for producing such material. Preferably, the material should be polymerized or at least pre-polymerized bioabsorbable compounds. Preferably, these compounds should be based on natural materials, especially proteinaceous materials, such as collagen, gelatine, fibrin or mixtures thereof.

### SUMMARY OF THE INVENTION

Accordingly, the present invention provides a method for the production of a polymerized product comprising the following steps:
providing a polymerization device to which a polymerization mixture and a separation medium can be applied and wherein flow of said mixture and medium can be conducted in appropriate ducts for said mixture and medium,
transporting said polymerization mixture in a duct of said polymerization device thereby allowing the polymerization reaction,
transporting said mixture in a duct of said polymerization device in a continuous flow,
interrupting said continuous flow of said mixture with said separation medium so as to obtain consecutive volumes of said mixture and volumes of said separation medium,
further transporting said consecutive volumes of said mixture and volumes of said separation medium in a duct of said polymerization device wherein said mixture further polymerizes to obtain a discontinuous polymerized product, and
removing said discontinuous polymerized product from said polymerization device.

According to another aspect, the invention relates to a method for the production of a fibrin product comprising the following steps:
providing a fibrinogen solution,
providing a thrombin solution,
providing a separation medium,
providing a fibrin polymerization device to which said fibrinogen solution, said thrombin solution and said separation medium can be applied and wherein flow of said solutions and medium can be conducted in appropriate ducts for said solutions and medium,
applying to said fibrin polymerization device said fibrinogen solution and said thrombin solution,
transporting said fibrinogen solution and said thrombin solution in ducts of said fibrin polymerization device and contacting said fibrinogen solution with said thrombin solution in the course of said transportation so as to obtain a homogeneous mixture of fibrinogen and thrombin and to allow the polymerization of fibrin,
transporting said mixture in a duct of said fibrin polymerization device in a continuous flow,

applying said separation medium to said fibrin polymerization device, transporting said separation medium in a duct of said fibrin polymerization device and interrupting said continuous flow of said mixture with said separation medium so as to obtain consecutive volumes of said mixture and volumes of said separation medium and wherein said mixture is polymerizing or already polymerized,
further transporting said consecutive volumes of said polymerizing or polymerized mixture and volumes of said separation medium in a duct of said fibrin polymerization device wherein said polymerizing or polymerized mixture optionally further polymerizes to obtain a discontinuous fibrin product, and
removing said discontinuous fibrin product from said fibrin polymerization device.

The invention also relates to polymerized or pre-polymerized products obtainable by such methods, especially collagen, fibrin and gelatine products; as well as to the polymerization devices used for producing the polymerized products according to the present invention.

### BRIEF DESCRIPTION OF THE FIGURES

FIGS. 1 to 3 show schematic representations of polymerization devices according to the present invention: FIG. 1 shows the section of the device where the polymerization mixture is separated by the separation medium into discontinuous volumes; FIGS. 2 and 3 show preferred embodiments of a fibrin polymerization device with fibrinogen and thrombin container;

FIG. 3 shows a hollow tool and a spine fusion cage for receiving the fibrin pearl product;

FIGS. 4 to 8 show various polymerized fibrin products with and without the ducts of the fibrin polymerization device according to the present invention;

FIG. 9 shows the general architecture of the air flow segmentation scheme;

FIGS. 10 to 13 show the results of experiments conducted with different flow rates and volumes for the polymerization mixture (fibrin) and the separation medium (air);

FIGS. 14a and b show withdrawal means for the separation medium; FIG. 15 shows in a control experiment that air bubbles are trapped into fibrin membrane; FIGS. 16a and b and 17 show fibrin products produced withdrawal means for the separation medium;

FIG. 18 shows the dependence of clotting times of Tisseel VH S/D from the activity of the thrombin solution; the clotting times at 37° C. of 1:1 mixtures of sealer protein- and thrombin solution was determined; three different lots were analyzed (each in triplicate).

FIG. 19 shows an experimental set-up using T-shaped connectors (“junctions”).

FIG. 20 shows Biorad™ connectors (“junctions”) and their performance in fibrin polymerization.

FIGS. 21 and 22 show Technicon™ connectors (“junctions”) and their performance in fibrin polymerization.

FIGS. 23a and 23b show production of segment volume depending on the ratio of flow rate Q.

FIG. 24 shows comparison of two configurations of segment volume depending on Q.

FIGS. 25 and 26 show the ratio surface/volume depending on the tubing intern diameter for different volumes of segments; theoretical values and formulae.

FIG. 27 shows fibrin polymers depending on the presence or the absence of a Mix-C device.

FIGS. 28a and 28b; 29a and 29b show Methylene blue release from fibrin segments into CaCl₂, over 14 days; error bars: std with n=3, numerotation: sample n°; samples at room temperature between measurements (FIGS. 28a and 28b); samples at kept refrigerated between measurements (FIGS. 29a and 29b).

FIGS. 30a and 30b show Doxorubicin release: percentage of doxorubicin present in the solvent depending on time error bars: std n=3; samples kept refrigerated between measures.

### DETAILED DESCRIPTION OF THE INVENTION

The present invention provides a method for producing polymers, especially biopolymers. The method provides a controlled polymerization reaction in a polymerization device in which the reaction is steered and operated.

The method for the production of a polymerized product according to the present invention comprises the following steps:
providing a polymerization device to which a polymerization mixture and a separation medium can be applied and wherein flow of said mixture and medium can be conducted in appropriate ducts for said mixture and medium,
transporting said polymerization mixture in a duct of said polymerization device thereby allowing the polymerization reaction,
transporting said mixture in a duct of said Polymerization device in a continuous flow,
interrupting said continuous flow of said mixture with said separation medium so as to obtain consecutive volumes of said mixture and volumes of said separation medium,
further transporting said consecutive volumes of said mixture and volumes of said separation medium in a duct of said polymerization device wherein said mixture further polymerizes to obtain a discontinuous polymerized product, and
removing said discontinuous polymerized product from said polymerization device.

The nature of the polymerization according to the present invention is not critical in principle; the size of the polymerization devices, the details in operating the systems and the provision of the polymerization products are mainly dependent on the nature of the polymerization reaction, especially the reaction kinetics, and can be adapted by a person skilled in the art for each of the polymerization reactions intended to be performed by the present invention. Of course, the faster the polymerization performs, the faster the process has to be conducted through the polymerization device according to the present invention. Accordingly, amounts and concentrations of the components of the polymerization mixture (preferably fibrinogen/thrombin; gelatine/thrombin; collagen/photoactivator; alginate/Ca²⁺) may be properly adjusted for each reaction set-up also depending on the desired properties of the polymerized material finally obtained. For example, the kinetics of the polymerization reaction of a given protein (e.g. collagen) may be adjusted to a specific crosslinker (e.g. DSS (disuccinimidyl suberate), BS3 (bis(sulfosuccinimidyl) 2,2,7,7-suberate-d4), Sulfo-SMCC (Sulfosuccinimidyl-4-[N-maleimidomethyl]cyclohexane-1-carboxylate)), SM(PEG) (Amine-to-sulfhydryl crosslinkers with soluble polyethylene), EDC (1-Ethyl-3-(3-dimethylaminopropyl)carbodiimide), Sulfo-NHS(N-hydroxysulfosuccinimide), glutathione, etc.). Other crosslinkers may be used, depending on the protein and on the polymerization reaction intended to be conducted (e.g. homo- or heterobifunctional crosslinkers, such as amine-to-amine (NHS(N-hydroxysuccinimide) esters (DSG, DSS, BS3, TSAT (trifunctional) (Tris-succinimidyl aminotriacetate)), NHS esters-PEG spacer (BS(PEG)₅, BS(PEG)₉), NHS esters-thiol-cleavable (DSP, DTSSP), NHS esters-misc-cleavable (DST (Disuccinimidyl tartrate), BSOCOES (Bis[2-(succinimidooxycarbonyloxy)ethyl]sulfone), EGS (ethylene glycolbis(succinimidylsuccinate), Sulfo-EGS), imidoesters (DMA (Dimethyladipimidate hydrochloride), DMP (Dimethyl pimelimidate.2HCl), DMS (Dimethyl suberimidate.2HCl)), imidoesters-thiol-cleavable (DTBP (Dimethyl 3,3′-dithiobispropionimidate-2HCl)), DFDNB (1,5-difluoro-2,4-dinitrobenzene), THPP (trifunctional) (β-[Tris(hydroxymethyl)phosphino] propionic acid), aldehyde-activated dextran); sulfhydryl-to-sulfhydryl (maleimides (BMOE, (bis(maleimido)ethane), BMB (bis(maleimido)hexane), BMH (bis(maleimido)hexane), TMEA (trifunctional) (Tris-(2-maleimidoethyl)amine)), maleimides-PEG spacer (BM(PEG)₂, BM(PEG)₃), maleimides-cleavable (BMDB(1,4-bismaleimidyl-2,3-dihydroxybutane), DTME), pyridyldithiols-cleavable (DPDPB), HBVS (vinylsulfone); nonselective (aryl azides (BASED-thiol-cleavable)); amine-to-sulfhydryl (NHS ester/maleimide (AMAS (N-[α-Maleimidoacetoxy] succinimide ester), BMPS(N-(β-Maleimidopropyloxy)succinimide ester), GMBS (MaleimidoButyryloxy-Succinimide ester) and Sulfo-GMBS, MBS (3-Maleimidobenzoyl-N-hydroxysuccinimide) and Sulfo-MBS, SMCC (Sulfosuccinimidyl-4-(N-maleimidomethyl)cyclohexane-1-carboxylate) and Sulfo-SMCC, EMCS and Sulfo-EMCS, SMPB and Sulfo-SMPB, SMPH (Succinimidyl-6-(B-maleimidopropionamido)hexanoate), LC-SMCC, Sulfo-KMUS), NHS ester/maleimide-PEG spacer (SM(PEG)2, SM(PEG)4, SM(PEG)6, SM(PEG)8, SM(PEG)12, SM(PEG)24), NHS ester/pyridyldithiol-cleavable (SPDP, LC-SPDP (Succinimidyl 6-[(3-2-pyridyldithio)propionamido]hexanoate) and Sulfo-LC-SPDP, SMPT, Sulfo-LC-SMPT), NHS esters/haloacetyl (SIA (N-Succinimidyl iodoacetate), SBAP (Succinimidyl 3-[bromoacetamido]propionate), SIAB (N-succinimidyl[4-iodoacetyl]aminobenzoate), Sulfo-SIAB), amine-to-nonselective (NHS ester/aryl azide (NHS-ASA (N-hydroxysuccinimidyl-4-azidosalicylic acid), ANB-NOS(N-5-Azido-2-nitrobenzyloxysuccinimide), Sulfo-HSAB (N-hydroxysulfosuccinimidyl-4-azidobenzoate), Sulfo-NHS-LC-ASA (sulfosuccinimidyl(4-azidosalicylamido]hexanoate), SANPAH and Sulfo-SANPAH), NHS ester/aryl azide-cleavable (Sulfo-SFAD, Sulfo-SAND, Sulfo-SAED), NHS ester/diazirine (SDA and Sulfo-SDA, LC-SDA and Sulfo-LC-SDA), NHS ester/diazirine-cleavable (SDAD and Sulfo-SDAD); amine-to-carboxyl (carbodiimide (DCC(N,N′-dicyclohexylcarbodiimide), EDC (1-Ethyl-3-(3-dimethylaminopropyl)carbodiimide))), sulfhydryl-to-nonselective (pyridyldithiol/aryl azide (APDP), sulfhydryl-to-carbohydrate (maleimide/hydrazide (BMPH, EMCH, MPBH, KMUH), pyridyldithiol/hydrazide (PDPH), carbohydrate-to-nonselective (hydrazide/aryl azide (ABH), hydroxyl-to-sulfhydryl (isocyanate/maleimide (PMPI)).

A person skilled in the art can adapt the present invention to the polymerization reaction intended, e.g. by applying the knowledge of kinetics, reaction conditions and flow rates of different polymerization mixtures to the polymerization reaction. Of course, the faster the polymerization occurs (e.g. due to higher concentration of crosslinker (e.g. thrombin concentration for example in the polymerization of fibrin), the higher the flow rate should be in order to move the polymerized material (e.g. fibrin) faster in the polymerization device, mainly for two reasons, to avoid to have the mixing unit clogged and to be able to cut the polymerized material properly with the separation medium). It is clear that the flow rate will not affect the polymerization time, however, determination of the flow rate of the polymerizing material together with the flow of the separation medium will determine the pattern of the segmented flow.

The process according to the present invention can be described best by the concept of liquid/liquid segmentation in a T-shaped junction. This is usually analysed in a model for drops formation in a microfluidic T-junction. The theory behind this phenomenon and its practical implications are e.g. disclosed by Amaya-Bower et al. (Phil. Trans. R. Soc. A 369 (2011), 2405-2413), De Menech et al. (J. Fluid Mech. 595 (2008), 141-161) and Thulasidas et al. (Chem. Eng. Sci. 50 (1995), 183-199). The “continuous phase” is the “continuous flow” according to the present invention (e.g. a liquid phase in a main channel before a T-junction), the “dispersed phase” is the interruption of the continuous flow with the separation medium according to the present invention (e.g. a phase that can enter the junction, for example perpendicularly to the “continuous” flow). For each fluid, key parameters are the flow rate Q (mL/min), viscosity p (cP), density ρ, and the interfacial tension between both fluids γ (mN/m). As in all fluid mechanics problems, it is also important to introduce dimensionless parameters which characterize each system (e.g. Reynolds number, capillary number, viscosity ratio and flow rates ratio).

The Reynolds number often gives important information about physics of a given system, however, for most microfluidic studies, the Reynolds number should always be ≦10 (for water as for air, for flow rates from 10⁻² to 10 mL/min and for T-junction width of around 1 mm), in a laminar flow.

The capillary number is particularly important in microfluidic systems. This number represents the effect of shear stresses versus the effect of interfacial stresses. Studies of droplets formation in a T-junction point out three regimes for the break up process which depend essentially on the capillary number of the flow: “squeezing”, “dripping and “jetting” regime (De Menech et al., 2008). The regimes differ with respect to the dynamics of breakup (“squeezing”: Buildup of pressure upstream of the emerging droplet; “dripping: Balance between interfacial and shear stresses exerted on the emerging droplet and buildup of pressure; and “jetting”: Balance between interfacial and shear stresses exerted on the emerging droplet (unbounded case)).

The dynamic of droplet breakup depends on the capillary number. When the capillary number is low (around <10⁻²), interfacial stresses dominate shear stresses, therefore the process of droplet formation is driven by the buildup of pressure upstream the droplet. In this regime, dynamics of breakup and droplet size are not (or not very much) influenced by the capillary number. On the other hand, when the capillary number is higher (>10⁻²), the system moves to a shear-driven mechanism of breakup. In this dripping regime, shear stresses are not negligible anymore and droplets size depends on the balance between interfacial stresses that opposes the growing of the droplet and shear stresses exerted on the emerging droplet by the continuous phase. Then, the jetting regime appears at very high capillary number (for high flow rates, very viscous fluids or low interfacial tension). This case is similar to the unbounded one. It is important to notice that the frontier between the squeezing and the dripping regime, in term of capillary number, depends on the viscosity ratio X between both fluids (De Menech et al., 2008).

For the present invention, it is preferred to use the “squeezing” regime, because it is easier to control. This phenomenon can be analysed and controlled by studying forces that are exerted on the tip of the dispersed phase (the interfacial tension force (F_{γ}), the shear stress force (F_{τ}) and the force which results of the drop of pressure upstream the tip (F_{R}). In applying the “squeezing” regime, the following order of magnitude for these three forces are obtained using the above considerations: F_{γ}=−γ·w (y=interfacial tension; w=width of the main channel (diameter of the duct)), F_{τ}=w·μ_{c}·Q_{c}/ε²(μ_{c}=viscosity of the continuous flow; Q_{c}=flow rate of the continuous flow; ε=thickness of the film of the continuous phase at the tip where the contact between the continuous phase and the dispersed phase is a thin film) and F_{R}=w²·μ_{c}·_{c}/ε². For preferred geometries according to the present invention w>>ε so that F_{τ}<<F_{R} and only F_{R} must be considered.

For a T-shaped junction in a squeezing regime, the size of a droplet is: V_{drop}=α+β.Q_{d}/Q_{c} (α, β: fitting parameters of order one, depend only on geometric parameters of the junction; Q_{d}=flow rate of the discontinuous flow). If Q_{d} is a gas inflow, it has to be considered that the gas is more compressible than a liquid and therefore the conditions in the ducts (e.g. pressure) have to be taken into account. It follows that for a small volume of bubbles, a linear scaling law can be used to describe the evolution of bubbles and drops size depending on the ratio of flow rates Q_{d}/Q_{c} when all other parameters are set. This law has been checked with experimental work in the experimental section. According to the present method a polymerization device is provided in which the polymerization reaction can be conducted. The mixture which should be polymerized is introduced into the device. Portions of polymerization mixtures are separated in the polymerization device according to the present invention by a separation medium. In the portions of the polymerization mixtures the polymerization reaction takes place (“is allowed”, i.e. reaction parameters are applied which allow polymerization reaction within these volumes separated by volumes of the separation medium) during conduct through the polymerization device. The separation medium safeguards that the polymerization portions are either completely separated or connected only by a small polymerization spacer (i.e. a section of the polymerized product with a significantly smaller volume of polymerized material, for example a fibrin film or fibrin membrane). The portions of the polymerization mixture separated by volumes of separation medium are transported in the polymerization device according to the present invention in ducts wherein the polymerization reaction is conducted, i.e. initiation of the polymerization reaction (e.g. by bringing two or more polymerization reaction partners together) and the subsequent partial or complete polymerization. The flow of the portions of polymerization mixture and separation medium is conducted continuously so that consecutive volumes of the polymerization mixture and separation medium are transported through the ducts of the polymerization device while the polymerization reaction takes place in the volumes of the polymerization mixture. With this process, a discontinuous polymerized product is obtained which can be removed from the polymerization device, if the desired degree of polymerization is obtained. Usually, the polymerization device and the process parameters are adjusted to the desired properties of the end product. For example, the inner diameter and the lengths of the ducts, the flow velocity or the time allowed for polymerization can easily be adjusted to the polymerization mixture and the desired properties (degree of polymerization, etc.) of the polymerized product to be obtained.

The method according to the present invention is preferably conducted with a polymerization mixture being a mixture of at least two components and which mixture is selected from a mixture of fibrinogen and thrombin, a mixture of gelatine and thrombin, a mixture of polysaccharide, especially alginate, and calcium, a mixture of polysaccharide and isocyanate, a mixture of poly(vinyl alcohol)-alginate and calcium, a mixture of albumin and aldehyde, a mixture of chitosan and glutaric dialdehyde, a mixture of chitosan and glycerol-phosphate disodium salt, a mixture of collagen and glutaraldehyde, a mixture of gelatin and glutaraldehyde, a mixture of polyethyleneglycol and amino acid with reactive end groups, a mixture of alginate—polyethyleneglycol diamines and carbodiimide.

Preferably, the polymerization device comprises at least one pressuring device for transporting mixture and medium, especially a pump or a plunger.

According to a preferred embodiment of the present method, the polymerization device comprises at least two containers for components of said polymerization mixture, said mixture being composed of at least two components.

The polymerization device preferably comprises a mixing device for the components so as to obtain said polymerization mixture. Preferred embodiments of the mixing device are a Y-shaped connector, a filter material, a three-dimensional lattice or matrix material.

The mixing device is preferably connected with the containers by ducts wherein the components (e.g. as solutions) can be transported the said container to the mixing device.

The present invention also relates to the polymerization device suitable for carrying out the method according to the present invention. A preferred polymerization device according to the present invention comprises—when operated—a polymer mixture which contains components selected from the group consisting of a biopolymer precursor, especially fibrinogen, thrombin, collagen, alginate, chitosan and mixtures thereof.

Preferably, at least one duct in the polymerization device according to the present invention contains withdrawal means for the separation medium to withdraw the separation medium.

The present invention is preferably used to provide a polymerized fibrin product made of a polymerization mixture comprising fibrinogen and thrombin. The enzymatic activity of thrombin cleaves fibrinogen to fibrin and fibrin monomers polymerize to a fibrin product (“fibrin aggregates”, “fibrin networks”, “fibrin clots”, “fibrin blocks”, “fibrin pearls”, “fibrin beads”, etc.) during the polymerization process. Presence of further substances in the polymerization mixture can influence the polymerization process (e.g. crosslinking agents, such as factor XIII) or provide advantageous properties of the resulting polymerized product (e.g. agents with pharmaceutical activity which are beneficial once the fibrin product is applied to a patient; e.g. antibiotics, growth factors, whole cells, genetic material, etc.).

In the method for the production of a fibrin product according to the present invention a fibrinogen solution, a thrombin solution and a separation medium are brought into a fibrin polymerization device wherein the flow of the fibrinogen solution, the thrombin solution and the separation medium can be conducted in appropriate ducts to allow the following actions: The fibrinogen solution and the thrombin solution are applied to the fibrin polymerization device and transported in the ducts of this fibrin polymerization device to allow the contact and mixture of the fibrinogen solution with the thrombin solution in the course of this transportation so as to obtain a homogeneous mixture of fibrinogen and thrombin. This homogeneous mixture of fibrinogen and thrombin results in a polymerization of fibrin. An efficient mixing of fibrinogen and thrombin upstream allows a uniform polymerization all over the volume of the polymerization mixture of fibrinogen and thrombin in the course of the further process. This polymerization mixture is transported in a duct of the fibrin polymerization device in a continuous flow.

A separation medium is also applied to the fibrin polymerization device and transported in a duct of the fibrin polymerization device. This separation medium then serves to interrupt the continuous flow of the fibrinogen/thrombin polymerization mixture and consecutive volumes of fibrinogen/thrombin polymerization and volumes of separation medium are obtained. In each of these segments of polymerization mixture fibrinogen is polymerizing or has already polymerized to a fibrin polymer.

The fibrin polymerizing flow is continuous upstream after mixture of fibrinogen and thrombin. E.g. the flow may be established from the junction with a tube conveying gas (SFA) or liquid (FIA). It is important to provide fibrin well and uniformly polymerized (one single phase) to be able to cut it with the gas or liquid flow. It is beneficial to prevent that the mixture is made of fibrin and free fibrinogen and thrombin (3 phases could result in this case because of bad mixing), because cutting with the separation medium (gas or liquid) may not be regular in such a case and shape of the resulting fibrin polymer (e.g. fibrin pearls) are less controllable in size and shape. It is therefore preferred to provide a mixing unit for fibrinogen and thrombin upstream of the junction with the duct conveying the separation medium (e.g. gas or liquid).

The degree of polymerization can easily be regulated by the provision of the reaction agents in the fibrin polymerization mixture. For example, the thrombin concentration can be used as a trigger for the polymerization degree of the final product. The degree of polymerization will mainly depend on the requirements of the surgeons for the specific surgical application. Accordingly, the residence time of the fibrin products formed inside of the duct is adjusted to these requirements as well as the thrombin concentration. It is well known that the adhesive property of fibrin depends on its polymerization state, fully polymerized fibrin does not stick on itself or on tissue but it is well polymerized so the ratio surface/volume is determined, the residence time/fibrinolysis can be easily monitored or controlled as well as the release of an active ingredient from it. If the fibrin is not fully polymerized or still liquid, it will spread all over the surface flatten, conform the tissue topology and stick to it. Two surfaces of tissue can be easily glued together. The timeframe for conducting the method according to the present invention is also depending on the fibrinogen concentration (at the same thrombin concentration, fibrinogen at low concentration will clot faster than at higher concentrations; although thrombin concentration is more critical as the higher the thrombin concentration, the faster polymerization occurs; temperature and pH can (i.a.) also be used to adjust polymerization rate to the rate intended and optimized for a given fibrin polymerization device).

The consecutive volumes of the polymerizing or polymerized mixture and volumes of the separation medium are further transported in a duct of this fibrin polymerization device wherein the polymerizing or polymerized mixture is optionally allowed to further polymerize to obtain a discontinuous fibrin product. Finally the resulting discontinuous fibrin product can be removed from the fibrin polymerization device.

In order to ascertain the continuous flow of the volumes through the fibrin polymerization device, this device preferably comprises at least one pressuring device for transporting the solutions and medium, especially a pump or a plunger. Preferably, actuation of liquid flow is implemented either by external pressure sources (gas cartridge), external mechanical pumps, integrated mechanical micro-pumps or by combinations of capillary forces and electrokinetic mechanisms. The driving force can be generated by utilizing piezoelectric, electrostatic, thermo-pneumatic, acoustic, electrocapillary, pneumatic or magnetic effects. It can also be a non-mechanical pumps function with electro-hydrodynamic, electro-osmotic or ultrasonic flow generation.

According to a preferred embodiment of the present invention, the polymerization device comprises separated containers for the fibrinogen solution, the thrombin solution and the separation medium. These containers can be easily exchanged or refilled as process chemicals without having to disrupt the fibrin polymerizing device as a whole.

Preferably, the mixing of fibrinogen and thrombin is effected by a mixing device which forms part of the fibrin polymerization device. According to a preferred embodiment, the mixing device is selected from the group consisting of a Y-shaped connector, a filter material, a three-dimensional lattice or matrix material. The mixing device can be connected with the containers for the fibrinogen and thrombin solution by ducts wherein these solutions can be transported from the containers to the mixing device.

The ducts of the fibrin polymerization device should preferably be made of a material which does not adhere (or only slightly adhere) to fibrin so as to allow a proper continuous flow of the volumes of polymerization mixture and separation medium. Preferably, the duct material is selected from the group consisting of Polyethylene (PE), High Density Polyethylene (HDPE), Polypropylene (PP), Ultra High Molecular Weight Polyethylene (UHMWPE), Nylon, Polytetra Fluoro Ethylene (PTFE), PVdF (polyvinylidene fluoride), Polyester, Cyclic Olefin Copolymer (COC), Thermoplastic Elastomers (TPE) including EVA.

(ethylene-vinyl acetate), Polyethyl Ether Ketone (PEEK), glass, ceramic, metal, synthetic and natural biodegradable biopolymers, hydro-biodegradable plastics (HBP) and oxo-biodegradable plastics (OBP), PHA (polyhydroxyalkanoates), PHBV (polyhydroxybutyrate-valerate), PLA (polylactic acid), PGA (Polygycolic acid), PCL (polycaprolactone), PVA (polyvinyl alcohol), PFT (polyethylene terephthalate), Polydimethylsiloxane (PDMS) or silicone rubber. The polymer which constitutes the duct can also be shape memory polymers (SMPs) or conductive polymers. The polymers can be treated by electrowetting or similar technique which is the modification of the wetting properties of a hydrophobic surface with an applied electric field.

The nature of the separation medium is not critical as long as it allows a proper separation of the volumes (segments) of the polymerizing mixture. It is preferably a gas or a liquid; however, it could also be solid, e.g. another polymer, especially a biopolymer, which does not mix with the fibrin product resulting from the fibrinogen/thrombin mixture. Preferably, the separation medium is selected from the group consisting of air, N₂, He, H₂, O₂, Ne, Ar, Kr, Xe, NO, NO₂, CO₂, N₂O, mixtures of such gases, H₂O, an aqueous solution, an organic solvent, media culture for growing cells, medical anaesthesia gases, such as entonox, nitronox or such gases mixed with air; fluorinated ether anaesthetics, such as sevoflurane, isoflurane, enflurane and desfurane; liquids having a higher density than the fibrin segment; insoluble liquids that can be supplemented with an active ingredient.

The separation medium can also be used for expelling the fibrin segments thereby making the fibrin polymerization device a zero dead volume device for fibrin, specifically in the case of air or another gas.

The fibrinogen solution and/or the thrombin solution may preferably further contain an additive (especially a pharmaceutically active additive), selected from the group consisting of Platelet Derived Growth Factor (PDGF) or Parathyroid Hormone (PTH), bone morphogenic proteins (BMP), hydroxypropylmethylcellulose, carboxylmethylcellulose, chitosan, photo-sensitive inhibitors of thrombin and thrombin-like molecules, self-assembling amphiphile peptides designed to mimic aggregated collagen fibers (extracellular matrices), factor XIII, cross-linking agents, pigments, fibers, polymers, copolymers, antibodies, antimicrobial agents, agents for improving the biocompatibility of the fibrin, proteins, anticoagulants, antiinflammatory compounds, compounds reducing graft rejection, living cells, cell growth inhibitors, agents stimulating endothelial cells, antibiotics, antiseptics, analgesics, antineoplastics, polypeptides, protease inhibitors, vitamins, cytokine, cytotoxins, minerals, interferons, hormones, polysaccharides, genetic materials, proteins promoting or stimulating the growth and/or attachment of endothelial cells on the cross-linked fibrin, growth factors, growth factors for heparin bond, substances against cholesterol, pain killers, collagen, osteoblasts, antimicrobial compositions, including antibiotics, especially tetracycline and ciprofloxacin.; antimycogenic compositions; antivirals, especially gangcyclovir, zidovudine, amantidine, vidarabine, ribaravin, trifluridine, acyclovir or dideoxyuridine; antibodies to viral components or gene products; antifungals, especially diflucan, ketaconizole and nystatin; antiparasitic agents, especially pentamidine; anti-inflammatory agents, especially alpha- or beta- or gamma-interferon, alpha- or beta-tumor necrosis factor; interleukins, drugs and mixtures thereof (i.e. more than one active compound). Of course, all proteinaceous compounds mentioned can be added from natural sources, but also from recombinant sources. Moreover, biological agents, such as a virus, bacteria, prion or fungus; human cells from endoderm, ectoderm and mesoderm as stem cells, endothelial osteoblast or chondrocytes cells from animal and vegetal sources can be added. Depending on the use of the separation medium (e.g. SFA or FIA) for the fibrin polymerizing device, the cells or additives will then be added in the fibrinogen solution when SFA technique is used OR in the liquid when FIA technique is used.

Preferred cell types to be included in the fibrin polymer produced by the method according to the present invention include: endoderm cells, especially gland cells (e.g. exocrine secretory epithelial cells), hormone secreting cells, ciliated cells with propulsive function; ectoderm cells, especially from the integumentary system (e.g. keratinizing epithelial cells or wet stratified barrier epithelial cells), from the nervous system (e.g. sensory transducer cells, autonomic neuron cells, sense organ and peripheral neuron supporting cells, central nervous system neurons and glial cells or lens cells), from mesoderm (metabolism and storage cells, barrier function cells (lung, gut, exocrine glands and urogenital tract) or kidney, extracellular matrix secretion cells, contractile cells, blood and immune system cells, pigment cells, germ cells, nurse cells or interstitial cells.

The polymerized fibrin product obtained by the method according to the present invention may be composed of separated fibrin polymer blocks (pearls, beads, etc.). However, according to a preferred embodiment of the present invention, the fibrin polymer volumes, i.e. the discontinuous fibrin products obtained are interconnected by polymerized fibrin material. The single blocks are then connected by a thin connection (fibrin film, fibrin thread, fibrin membrane, etc.) similar to a pearl necklace (“fibrin pearls”). This product is flexible but nevertheless shows a connectivity of the separate blocks. The fibrin pearls can therefore be applied e.g. during surgery until the volume needed is reached (whereafter the connection may easily be cut). The administered fibrin blocks are still interconnected (and will not immediately separate from each other) which makes them much easier to handle during administration than individual single blocks. Accordingly, such a preferred discontinuous fibrin polymer product according to the present invention consists of separated volumes of polymer material corresponding to the consecutive volumes of said polymerized mixture and being connected by a (relatively) thin interconnecting polymer section. The general process to produce either a continuous product (e.g. a “fibrin necklace”) or a discontinuous product (e.g. separated “fibrin pearls”) is usually the same. Often, only a flange or a similar device will be required as device feature to generate separated fibrin pearls. Examples for the generation of continuous or discontinuous polymers according to the present invention are also disclosed in the example section of the present application.

In the fibrin polymerization device according to the present invention, the duct transporting the polymerizing mixture of fibrinogen and thrombin and the duct transporting the separation medium are preferably connected by a T- or Y-shaped connector.

Preferably, the ducts and/or connectors have an internal diameter of 0.2 to 5 mm, preferably from 0.6 to 2 mm, especially of 1.2 to 1.6 mm. It is clear the provision of different tubing (ducts) having different internal diameters generate different pearl size. Different pearl size will have different surface/volume ratio that will give different pharmakinetic release profile. At the same flow rate of fibrin and e.g. gas, the size of the fibrin pearls will be different depending on the internal diameter the tube. For example, if the gas liquid flow rate thumb pressure is controlled electronically, a determined and defined number of fibrin pearls may be generated. If the number is controlled, volume is known, surface is known so the pharmacokinetic release of the active ingredient can be easily predetermined and adjusted.

According to a preferred embodiment of the present invention, the duct wherein the consecutive volumes of the polymerizing or polymerized mixture and volumes of the separation medium are transported in the fibrin polymerization device contains withdrawal means for the separation medium to withdraw the separation medium. These withdrawal means for the separation medium can e.g. be holes or semipermeable surfaces in the duct or absorption devices for the separation medium in the duct. For example, it is easy to remove the air pocket (if the separation medium is air or another gas) by making (one or more (e.g. two, three, four, five, ten, or more)) holes at the distal part of the duct. In addition to such holes, “flanges” can be used in this connection. For example, such flanges can be internally generated when making the holes and contributing to cut the fibrin membrane formed between the polymerization volumes. Ideally, (a) couple(s) of flanges is generated by appropriate holes opposite to each other. For example, flange dimensions may be ranging from 0.05 to 0.5 mm. Instead of or in combination with holes, (a) ring(s) equipped with pins that are piercing the plastic tube may be used.

In fibrin polymer product obtained by the present method air pockets may be trapped in a fibrin membrane. Although this could be advantageous in some instances, for other instances such air pockets are less desirable. For example, the space of the spine fusion cage could be filled out with air at the expense of the pre-polymerized fibrin material. In order to avoid the presence of air pockets that are mandatory for cutting the polymerizing fibrin polymer product according to the present invention, air vents (as separation medium withdrawal means) can be provided on the distal part of the container that is used for temporary storing fibrin. The air vents can be holes with dimension that depends on the amount of air, flow rate, length and diameter of the tube, degree of polymerization of fibrin, mechanical properties of the fibrin membrane formed around the air bubble, surface tension of the inner wall of the container, etc.

The number, shape and position of the holes can be determined according to the biological and physical requirements described above. It can be a simple hole made by a core leaving or not container flashes around the holes in contact with the material conveyed into the tube. It can be a tip to the distal end of the tube for example containing two needle pins that are fitting with holes or a tip with pins that are puncturing the tube during the assembly process.

According to a preferred embodiment, the method according to the present invention is conducted in a segmented flow analysis (SFA) format or in a flow injection analysis (FIA) format. Basically a device using SFA will look the same as the device using the FIA (air (gases) will be replaced by a liquid to segment the fibrin flow). Volume of air container could be smaller than the air container for FIA which results in a smaller device.

One big advantage of air or other gases is their compressibility while liquids, such as water cannot be compressed. Segmented flow analysis (SFA) uses air segmentation to separate a flowing stream into numerous discrete segments to establish a long train of individual samples moving through a flow channel, while flow injection analysis (FIA) separates each sample from subsequent sample with a carrier (liquid) reagent. A person skilled in the art is well aware of the principles of these methods and how to apply them in practicing the present invention. For example, general principles of SFA may be derived from the following sources: Gardner et al., Anal. Chem., 1983, 55 (9), pp 1645-1647; Begg, Anal. Chem., 1971, 43 (7), pp 854-857; ASTM D7511-09e2 Standard Test Method for Total Cyanide by Segmented Flow Injection Analysis, In-Line Ultraviolet Digestion and Amperometric Detection; Roman et al., Anal Chem. 2008 Nov. 1;80(21):8231-8; 4120 Segmented Continuous Flow Analysis approved by Standard and Method, SM Committee: 1997; general principles of FIA may be derived from the following sources: Ranger, Anal. Chem., 1981, 53. (1), pp 20A-32A; Hansen, J Mol Recognit. 1996 September-December;9(5-6):316-25; Ruzicka et al., Anal. Chem., 1991, 63 (17), pp 1680-1685; Ashish et al., J. Chem. Pharm. Res., 2010, 2(2): 118-125.

As already mentioned, the dimensions of the ducts may be adjusted to the reaction parameters and the properties of the desired end product. The faster the polymerization reaction occurs (e.g. the higher the thrombin concentration is), the shorter the ducts can be provided. For example, in the fibrin polymerization device according to the present invention, the ducts used have a preferred individual length of 1 mm to 10 m, more preferred from 0.5 cm to 3 m, especially from 1 to 50 cm. From such preferred dimensions, the preferred volumes of the polymerizing or polymerized mixture sections (i.e. the sections within two volumes of separation medium) is from 0.5 to 20 μl, more preferred from 1 to 5 μl.

According to a preferred embodiment, the duct(s) can be the inner volume of a surgical tool, shaft or holder. The device could then be directly connected to the surgical tool which would simplify administration during surgery. Similarly, the duct (at the rear end of the fibrin polymerization device) can be connected to a medical (or not), implantable (spine fusion cage) or non-implantable device. Also the velocity of transporting these volumes continuously through the fibrin polymerization device can be adjusted to the desired final fibrin polymer product. With the dimensions above and typical thrombin and fibrinogen concentrations (preferred thrombin/fibrinogen concentrations to be used in the present method are 0.1 to 5000 I.U. thrombin/ml, preferably 4 to 3000, more preferred 10 to 1000, especially 50 to 500 and/or 50 to 150 mg fibrinogen/ml, preferably 70 to 120, especially 80 to 100, more preferred 4), suitable transporting velocities (flow rates) may be in the range of 0.05 to 50 ml/min, preferably of 0.5 to 20 ml/min, especially of 1 to 10 ml/min.

At the end of the polymerization, the final product may be freed from the surrounding (rear end of the) duct; it may also be kept in the duct as a storage device. In some instances it is preferred to remove the duct wherein the discontinuous fibrin product is present.

Polymerization temperature may be an important process parameter for many polymerization reactions. For example, temperatures of 10 to 50° C. or 30 to 40° C., especially about 37° C. may be beneficial for fibrin polymerization. It may therefore be advantageous to provide heating and/or cooling means in the fibrin polymerization device for heating and/or cooling at least parts of this fibrin polymerization device, especially ducts or containers for allowing temperature control of the polymerization reaction and of the starting and end products (e.g. the fibrinogen and thrombin solution or the resulting fibrin polymer product).

The finally obtained fibrin product may then either be directly applied to a patient or—what will usually be the case—can be brought into a storage form, e.g. by suitable packaging. A preferred form for storing fibrin polymer products is storing them in a lyophilized state. This usually enables a significantly increased storage time. It is therefore a preferred embodiment of the present invention to lyophilize the final fibrin product after removing from said fibrin polymerization device.

According to another aspect, the present invention concerns the novel fibrin polymer products obtainable by the method according to the present invention. A specifically preferred embodiment of these novel fibrin polymer products according to the present invention are the “fibrin necklace” structures obtained if a fibrin connection between the fibrin polymer volumes is provided. In general, it is possible to make either separated fibrin pearls or to provide them in a fibrin necklace form. For example, a membrane can be allowed to form along the inner wall of the air segment (e.g. in the SFA technique) which is linking the fibrin pearls together to make a fibrin necklace. Pearl separation can be promoted or membrane formation avoided when polymerization is fast (e.g. by a high thrombin concentration). At the “T” junction (e.g. (immediately) after mixing), fibrin obtained is liquid enough to be cut by a separation medium bubble then when the fibrin segment is formed polymerization reached a level where most of the fibrinogen is involved into the polymerization of the fibrin segment. Even if there are traces of thrombin on the inner wall of the tube, there is not enough fibrinogen to form a membrane on it. For example with thrombin concentration ranging from 20 up to 1000 IU/ml and more the membrane can be avoided. On the contrary, when the polymerization is slow and continues after the addition of the separation medium, thrombin will adsorbed on the inner wall of the tube and free fibrinogen still present into the fibrin/fibrinogen segment will react with the thrombin and form the membrane. As already mentioned, “fibrin pearl necklace structures” are specifically advantageous in surgical practice due to the flexibility of the product as well as due to the fact that the fibrin pearls are Still interconnected (and therefore controllable compared to the dispersing character of non-interconnected fibrin pearls). In this connection it is an advantageous embodiment to fill the air/membrane section with another biopolymer, e.g. by using the other biopolymer as a separation medium.

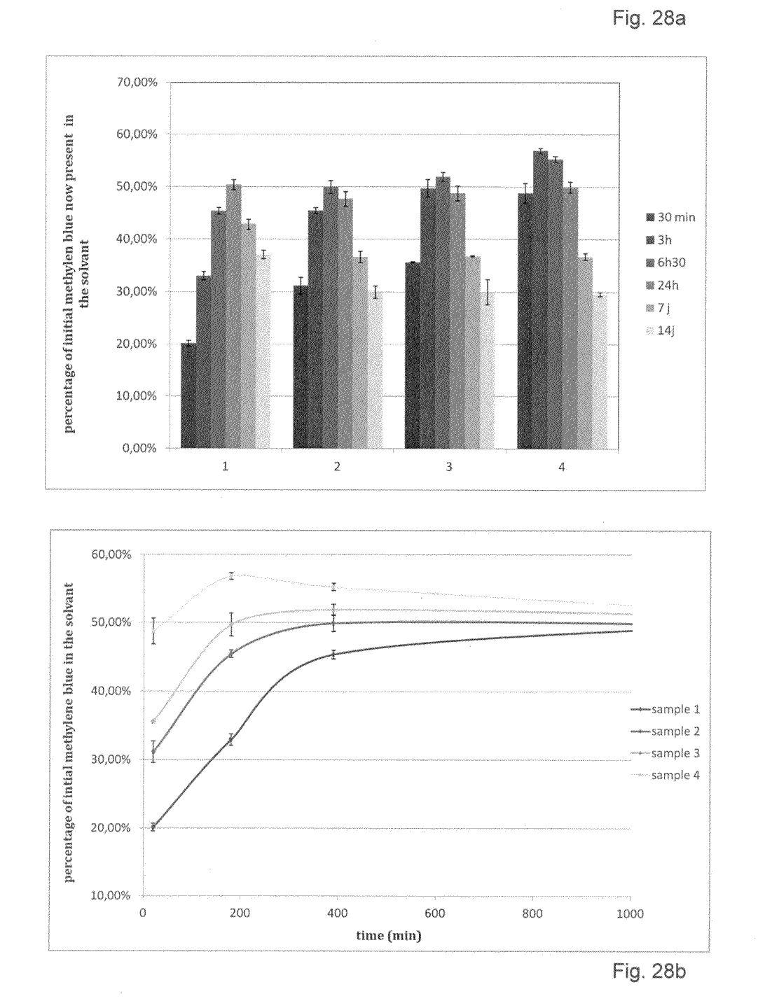

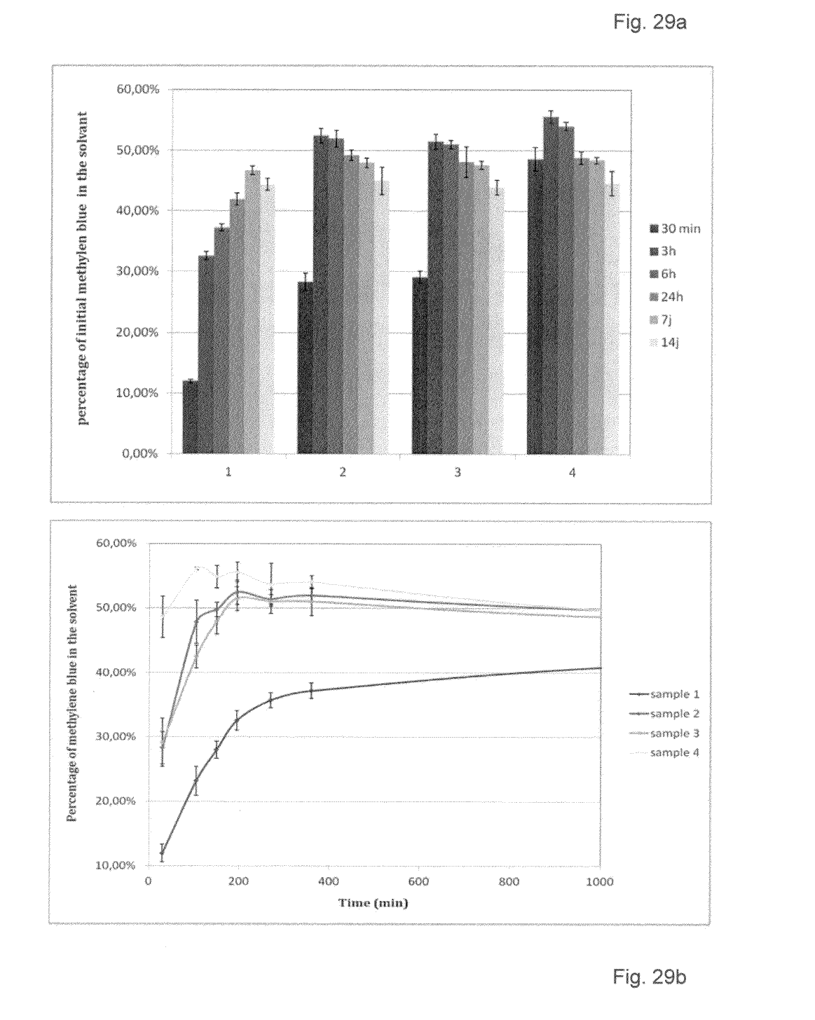

A specifically preferred fibrin product according to the present invention is a product which is characterized by a uniform size of the fibrin blocks that are produced. Whereas the device according to the present invention may be worked to produce fibrin polymer blocks of irregular sizes, it is preferred to perform the process according to the present invention in a mode where the shape and size of the resulting fibrin polymer blocks is regular and uniform. This can be achieved by controlling the production under a “squeezing” regime. It is possible to adjust the process parameters of the present method according to the teachings presented in the present application so as to obtain regularly shaped fibrin polymer block. For example, final fibrin products can be obtained wherein—in a preparation of at least 20, at least 100, at least 1000, at least 10 000 fibrin blocks—more than 80% of the fibrin blocks obtained have the same volume (the “same volume” meaning within only 20% or less deviation from the mean volume). Low deviation and accurate fibrin segment length and volume are shown in FIGS. 28a and 29b on the low deviation over time for different samples.

Another special fibrin polymer according to the present invention is obtainable by the use of withdrawal means for the separation medium. The fibrin product can then be compressed and put into a storage stable form in the duet of the fibrin polymerization device. Removal of the separation medium may then be advantageous if the separation medium does not play any role for administration of the fibrin product (e.g. if the separation medium is only air or a water and does not contain e.g. a pharmaceutically effective agent which assists the administration of the fibrin product during surgery).

The fibrin product which is finally obtained by removing the duct of the fibrin polymerization device may be compressed to obtain a storage form (or dried (lyophilized)).

The fibrin product according to the present invention may be made from natural sources (e.g. plasmatic fibrinogen and plasmatic thrombin), however, use of recombinant process components (e.g. recombinant fibrinogen and/or recombinant thrombin and or recombinant factor XIII, etc.) is also possible. The use of recombinant proteins is specifically advantageous e.g. to control contaminants, cost, availability of proteins, etc.

The final fibrin polymer product according to the present invention can also be treated by virus inactivation treatments, especially preparation in an aseptic environment like in an operating room for the partially or fully polymerized fibrin segments (e.g. preparation in an aseptic environment, β irradiation, y irradiation at 10 Mrad, ebeam on the fully polymerized fibrin segments or on the freeze dried fibrin segments). The final, optionally virus inactivation treated fibrin product is then stored in a suitable package, e.g. in a sterile container.

The fibrin polymerization device according to the present invention indeed is a general polymerization device which can be used for all polymerization reactions. Specifically for the production of biopolymers, the present device can easily be adapted from fibrin polymerization to other biopolymers, such as alginate, collagen, gelatine, chitosan, hyaluronic acid, etc. or mixtures thereof. Usually, only slight modifications have to be performed to change the polymerization product in a given polymerization device (besides, of course, the provision of specific polymerization mixture and the separation medium. The present invention is also specifically suited for providing foamy polymers, e.g. fibrin foams, gelatine foams or collagen foams. For providing foam products according to the present invention, a gaseous medium can be applied to the polymerization mixture (or at least to one of the components of the polymerization mixture before to be foamed with one or more porous material and mixed with the second components) and then subjected to the separation medium. Alternatively, the foamed polymerization mixture (made e.g. by swooshing of the polymerization mixture) can be directly applied to the polymerization device according to the present invention.

The polymers according to the present invention are specifically suitable in dental, gynecologic, urologic, ophthalmologic, trauma, head and neck, neurosurgery, cardiac, thoracic, oncologic, plastic and drug delivery surgery as well as in tissue engineering, for filling soft tissue defects, hard tissue defects and implantable devices. For example, the polymers according to the present invention can be used as dermal fillers: Collagen is probably one of the most popular dermal fillers because of the excellent results obtained and because it is a natural protein which supports the skin even after the polymerization process according to the present invention. Hyaluronic acid is another natural substance that is found within the human bodies. Hyaluronic polymers made according to the present invention are preferably used to provide fuller lips and fill scars and for moderate to severe folds and wrinkles. The polymer fillers according to the present invention can e.g. be injected into the skin as dermal filler (see further: Ascher et al., Ann. Chir. Plast. Esthet. 2004 October;49(5):465-85; WO2010/003104A; Fodor, Plastic and Reconst. Surg. 88 (2) (1991), 382; Kozluca et al., Art. Organs 19 (9) (1995), 902-908; U.S. Pat. No. 7,935,361A, U.S. Pat. No. 7,790,194A, U.S. Pat. No. 7,011,829A).

The fibrin pearls according to the present invention can be used in all applications that are currently using fibrin glue to fill cavities, defects, spaces, etc., even in indications where fibrin has to be injected (here, a tube diameter can be provided that matches the gauge of the needle). If the pearls have to stick to the surrounding tissue, full polymerization of fibrin can be avoided in order to get it sticky to the tissue. In that case the fibrin pearls can be used in combination of the regular fibrin glue which is applied before the application of the pearls. This could e.g. be done by a single device in two steps: Step 1, no separation medium is applied (valve blocks the SM channel before the T junction); Step 2, the separation medium is delivered. As already stated, the production of fibrin polymers is a specifically preferred embodiment of the present invention. However, the present invention is obviously not limited to the polymerization of fibrin from fibrinogen and thrombin. This principle can be applied to many other biological polymerization processes wherein biological polymers are obtained by a controllable polymerization process wherein the polymerization partners can be transported while polymerization reaction continues. Such biological polymers have been proven to be advantageous in principle, especially due to their bioabsorbability properties. In principle, any microreactor set-up can be used for such reactions. The dimensions and process parameters can be easily adjusted to the nature of the polymerization reaction and the aimed properties of the resulting polymerized products.

As already mentioned, other specifically preferred polymerization reactions to be conducted by the present invention are the gelatine/thrombin and collagen/photoactivator process, wherein the same principles as described above for the fibrin polymerization may be applied. Other combinations include a mixture of polysaccharide, especially alginate, and calcium, a mixture of polysaccharide and isocyanate, a mixture of poly(vinyl alcohol)-alginate and calcium, a mixture of albumin and aldehyde, a mixture of chitosan and glutaric dialdehyde, a mixture of chitosan and glycerol-phosphate disodium salt, a mixture of collagen and glutaraldehyde, a mixture of gelatin and glutaraldehyde, a mixture of polyethyleneglycol and amino acid with reactive end groups, a mixture of alginate polyethyleneglycol diamines and carbodiimide. The present invention is specifically suited to provide mixtures of such polymers, such as mixtures of fibrin and collagen, fibrin and gelatine, gelatine and collagen, fibrin and alginate, collagen and alginate, fibrin and gelatine and collagen, alginate and fibrin and gelatine, chitosan and alginate and fibrin, gelatine and chitosan and alginate and fibrin, etc. With the present method and device, these mixtures are much easier to prepare than with standard proceedings which include mixing of the components without the present transport and polymerization process.

For example, gelatin-resorcin-formalin glue widely used in the surgical treatment of dissecting aneurysms, and especially in acute aortic dissection type A is disclosed in Fukunaga et al., Eur J Cardiothorac Surg 1999; 15:564-570. Enzymatic cross-linking versus radical polymerization in the preparation of gelatine PolyHIPEs and their performance as scaffolds in the culture of hepatocytes is disclosed in Barbetta et al. (Biomacromolecules. 2006 November; 7(11):3059-68) wherein it is described that two different cross-linking procedures were adopted: (I) radical polymerization of the methacrylate functionalities, previously introduced onto the gelatine chains and (II) formation of isopeptide bridges among the gelatine chains promoted by the enzyme microbial transglutaminase; the method of cross-linking exerts a pronounced effect on the morphology of the porous biomaterials: radical polymerization of methacrylated gelatine allowed the production of scaffolds with a better defined porous structure, while the enzymatically cross-linked scaffolds were characterized by a thinner skeletal framework. Gelatine hydrogel prepared by photo-initiated polymerization and loaded with TGF-beta1 for cartilage tissue engineering is disclosed in Hu et al., Macromol Biosci. 2009 Dec. 8; 9(12):1194-201; in this work, the gelatine molecule was modified with methacrylic acid (MA) to obtain crosslinkable gelatine (GM), which formed a chemically crosslinked hydrogel by photoinitiating polymerization; the gelation time could be easily tuned and showed an inverse relationship with the GM concentration. The facile preparation of pH-responsive gelatine-based core-shell polymeric nanoparticles at high concentrations via template polymerization is disclosed by Zhang et al., Polymer Volume 48, Issue 19, 10 Sep. 2007, Pages 5639-5645, reporting that the structure stability of the nanoparticles was improved by selectively crosslinking gelatine with glutaraldehyde. Gelatine polymerization vs. low molecular weight dextran (gasometric and hemodynamic variations in cesarean section) is disclosed by Tamayo et al., Ginecol Obstet. Mex. 1975 November; 38(229):391-401. Encapsulation of Chondrocytes in Photopolymerizable Styrenated Gelatin for Cartilage Tissue Engineering is disclosed by Hoshikawa et al., Tissue Engineering August 2006, 12(8): 2333-2341, reporting that a photopolymerizable styrenated gelatine was developed that can cross-link through polymerization induced by irradiation with visible light. Martineau et al. (Defence Research and Development Canada http://pubs.drdc.gc.ca/PDFS/unc48/p524644.pdf) disclose the process for photo cross-linking the components of a biopolymer-elastomer interpenetrating polymer network (IPN) biomaterial for use as a wound dressing; cross-linking of methacrylated gelatine was performed by ultraviolet irradiation in the presence of a photoinitiator.

With respect to collagen, e.g. Evans et al. (Biochem J. 1983 Sep. 1; 213(3): 751-758) report the promotion of collagen polymerization by lanthanide and calcium ions; Ca²⁺ (1-5 mM) and lanthanide (20-250 microM) ions enhanced the rate of polymerization of purified calf skin collagen (1.5 mg/ml) at pH 7.0 in the presence of 30 mM-Tris/HCl and 0.2 M-NaCl. Collagen Cross-Linking (CCL) can be obtained by using Riboflavin and UV (365 nm) exposure or C3R; collagen crosslinking by the photosensitzer riboflavin and ultraviolet A-light is disclosed as an effective means for stabilizing the cornea in keratoconus. The predominant chemical agent that has been investigated for the treatment of collagenous tissues is glutaraldehyde, which gives materials with the highest degree of crosslinking when compared with other known methods such as formaldehyde, epoxy compounds, cyanamide and the acyl-azide method. Even beyond biopolymers, the present invention may be applied to any controllable polymerization process wherein polymers are obtained by a controllable polymerization process and wherein the polymerization partners can be transported while polymerization reaction continues. Even in quicker polymerization processes, the transportation in the ducts allows a proper obtaining of continuous polymerization products, provided that the dimensions of the polymerization device and the process parameters, especially the continuous flow velocity, is properly adjusted to the polymerization reaction. For example, faster polymerization reactions could be handled in smaller polymerization devices (e.g. microreactors) or faster flow rates. On the other hand, slower reactions may be controlled by slower flow rates. For example, alginates, cyanoacrylates, polyurethanes, epoxy glues, acrylic and cyanoacrylate adhesives or other sealants may be applied as well as albumin, polysaccharides (e.g. chitosan), hyaluronic polymers, starch or other examples (such as the ones disclosed in U.S. Pat. No. 5,880,183 A).

The cross-linking mechanisms of calcium and zinc in production of alginate microspheres is disclosed by Chan et al., Int. J. Pharmaceutics 242 (1-2) (2002), 255-258, where calcium chloride and zinc sulphate were used to cross-link alginate microspheres prepared by an emulsification method. The microspheres cross-linked by a combination of these two salts showed different morphology and slower drug release compared with those cross-linked by the calcium salt alone. In Hillgärtner et al. (Eur. Biophys J (2004) 33: 50-58) the cross-linking properties of alginate gels determined by using advanced NMR imaging and Cu²⁺ as contrast agent were disclosed. For the formation of empty alginate microcapsules an air-jet two-channel droplet generator was used. The inner channel (0.5 mm in diameter) contained the alginate solution, the second one fed the air supply into the nozzle. The injection rate of the alginate into the nozzle was controlled by an electric motor. Homogeneous alginate droplets with diameters of between 400 μm and 6001m were generated by regulating the velocity of the co-axial air stream. The droplets entered a bath solution containing multivalent cations to induce cross-linking. The formation of alginate microspheres produced using emulsification technique was disclosed by Heng et al. (J. Microencapsul. 2003 May-June;20(3):401-13) wherein the alginate microspheres were produced by cross-linking alginate globules dispersed in a continuous organic phase using various calcium salts: calcium chloride, calcium acetate, calcium lactate and calcium gluconate.

Instant Adhesives (Cyanoacrylate adhesives) are generally disclosed in Vol2 of the homonymous document by Three Bond Technical News (Issued Jun. 20, 1991, 34); the main components of instant adhesives, 2-cyanoacrylate (2-cyanoacrylic acid ester), feature two strong electron attracting groups—the cyano group (—CN) and the carbonyl group (C═O)—on a single carbon atom in the vinyl group (CH2=C—) Thus, this substance reacts readily with relatively weak nucleophilic solvents (Nu—) such as water and alcohol, curing through polymerization.

Petrie (“Handbook of Adhesives and Sealants” published by MacGraw-Hill) reviews epoxy, polyurethane, acrylic, and cyanoacrylate adhesives, especially the families of polymeric materials that are most often employed in structural adhesive formulations (epoxy, epoxy-hybrid, polyurethane, acrylic, and cyanoacrylate adhesives).

Other sealants include sealants, such as DuraSeal [Confluent Surgical Inc., Waltham, Mass., USA], BioGlue [Cryolife, Kennesaw, Ga., USA], KiOmedine (KitoZyme S. A, Belgium), BioGlue (Cryolife, Atlanta, USA), GPS III (biomet, Warsaw, USA); fibrin glues, such as (EVICEL [Johnson and Johnson Wound Management, Ethicon Inc., Somerville, N.J., USA], Quixil® [Johnson and Johnson Wound Management, Ethicon Inc., Somerville, N.J., USA, Tisseel [fibrin sealant; Baxter International Inc., Westlake Village, Calif., USA]), Artiss [fibrin sealant; Baxter International Inc., Westlake Village, Calif., USA], CoStasis [Cohesion Technologies, US Surgical, which combines bovine collagen and bovine thrombin with autologous plasma obtained in a centrifugation process from the patient], Crosseal [American Red Cross, Washington, D.C.], CryoSeal AHS [Thermogenesis, Sacramento, Calif.; a computerized system capable of cryoprecipitating human fibrinogen], ReliSeal®, Beriplast [Centeon, Marburg, Germany], Biocol [Bio-transfusion, Lille, France], Haemocomplettan [Centeon], Hemaseel APR [Haemacure, Inc., Quebec, Canada], Hemaseel HMN [Haemacure, Inc., Quebec, Canada]; albumin sealants, such as PoliPhase® Surgical Sealant [from Avalon Medical; comprised of serum albumin substrate and heat stabilized aldehyde crosslinker; crosslinking of proteins with aldehydes typically takes place via Schiffs base chemistry in which primary and secondary amines are covalently attached to the carbonyl functionalities of the crosslinker]; polysaccharides, such as chitosan (e.g. Hsien et al (Separation Science and Technology, 1520-5754, Volume 30, Issue 12, 1995, Pages 2455-2475) disclose the effects of acylation and crosslinking on the material properties and cadmium ion adsorption capacity of porous chitosan beads: chitosan is described as a novel glucosamine biopolymer derived from the shells of marine organisms and heterogeneous crosslinking of linear chitosan chains with the bifunctional reagent glutaric dialdehyde (GA). Hyaluronic biopolymers are disclosed by Kogan et al. (Biotechnol Lett. 29(1) 2007:17-25). Other examples are e.g. disclosed in U.S. Pat. No. 5,880,183 A wherein a composition of a hydroxyl functional polymer, an acetate functional polymer and a carboxyl functional polymer are disclosed, which are crosslinked by a polyfunctional aziridine. Preferably, PVOH, polyvinylacetate and a carboxylated styrene/butadiene are used as the polymers.

Alternative natural polymers specifically recommended for bone regeneration are starch-based polymershyaluronan, hyaluronan, and poly(hydroxyalkanoates). Starch is a carbohydrate consisting of a large number of glucose units joined together by glycosidic bonds. Starch-based polymers have been demonstrated to be potentially useful for tissue engineering of bone because of their interesting mechanical properties.

The polymers according to the present invention, especially the fibrin polymers, may contain additives. Preferred additives for use in the odontoiatric and plastic surgery field are e.g. disclosed by Bressan et al. (Polymers 2011, 3, 509-526). Bressan et al. disclose that calcium orthophosphates are interesting hard tissue engineering biomaterials because of their similarity to the mineral component of mammalian bones and teeth. Nanohydroxyapatite based products now commercially available for bone filling, are NanOss, Ostim and Vitoss. NanOss is a bone filler from Angstrom Medica considered to be the first nanotechnological medical device. It is mechanically strong and osteoconductive. Ostim is a ready-to-use injectable bone matrix in paste form. Vitoss, a beta-tricalcium phosphate bone, is clinically suitable as a filler. The invention is further illustrated in the following examples and the drawing figures, yet without being limited thereto.

The polymerization device according to the present invention has been described above specifically for the use as fibrin polymerization device, however, the present device is suitable for carrying out virtually any polymerization reaction, preferably polymerization reactions providing biopolymers, especially biocompatible or biodegradable biopolymers for use in human surgery.

It is therefore preferred to provide the present polymerization device as a biopolymer production device. Therefore, a suitable polymer mixture contains a biopolymer precursor, especially fibrinogen, thrombin, collagen, alginate, chitosan or mixtures thereof.

According to another aspect, the present invention also provides a kit for assembling polymerization devices according to the present invention, said kit comprising ducts, preferably ducts with two or more different inner diameters, at least one polymer mixture inlet, at least one separation medium inlet and at least one flow device. With such a kit, suitable polymerization product can be designed and nature, shape, diameter, etc. of the product can easily be changed by replacing the parts of the devices with other parts, e.g. replacing a duct with a certain diameter with ducts with another diameter to obtain differently sized polymer pearls or necklaces.

The kit according to the present invention preferably comprises all mandatory features of the present polymerization device, especially with more than one embodiment of such features. Additionally, the kit may contain preferred parts of the polymerization device, for example one or more polymerization mixture preparation device(s), at least one duct with holes and/or flanges, polymerization mixture components, preferably fibrinogen, thrombin, collagen, alginate, chitosan, at least one metal ion preparation, at least one photoactivator or mixtures thereof, provided that the mixture does not already constitute a polymerization mixture.

### EXAMPLES

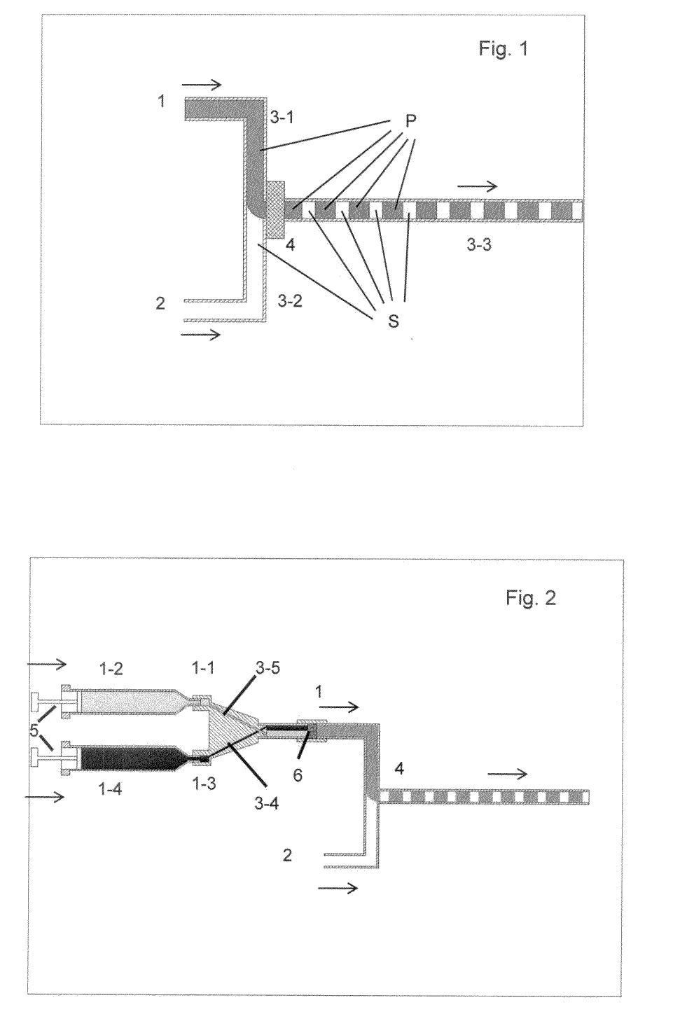

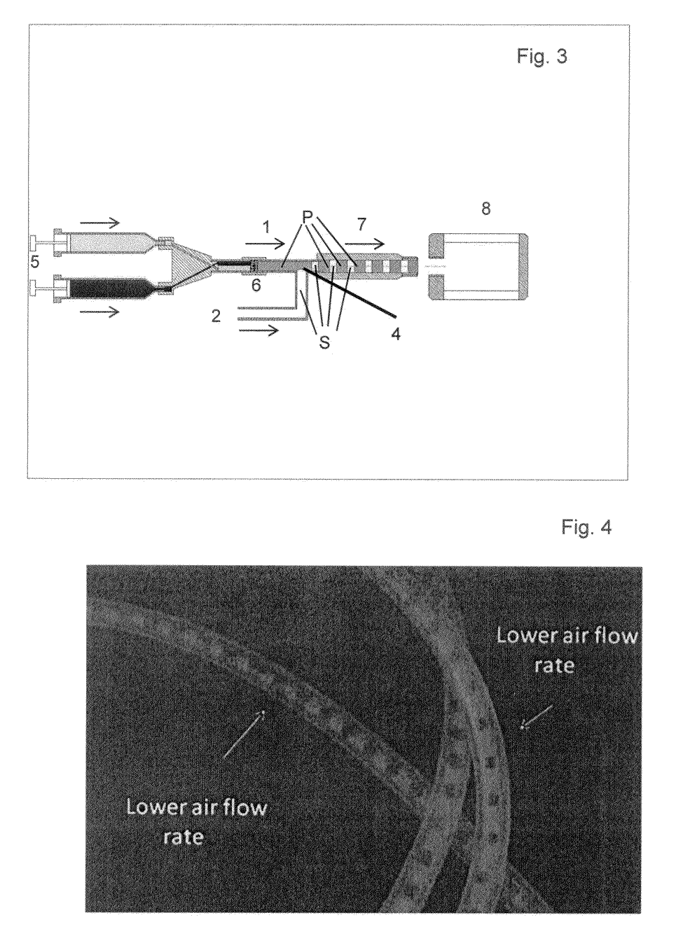

FIGS. 1 to 3 show schematic representations of polymerization devices according to the present invention. FIG. 1 shows the central features of the device: an inlet (1) for the polymerization mixture (e.g. a mixture of a fibrinogen solution and a thrombin solution) and an inlet (2) for a separation medium; ducts for conducting flow and transport of the mixture and the separation medium and the polymerizing products (3-1,3-2, 3-3); means for interrupting the continuous flow of the mixture with the separation medium (4). In FIG. 2 an inlet for a fibrinogen solution (1-1) and a container for a fibrinogen solution (1-2), an inlet for a thrombin solution (1-3) and a container for the thrombin solution (1-4) are shown as a double syringe system (e.g. a Duplojec® device). The plungers (5) act as pressuring devices for transporting the solutions through the device. The mixing device for fibrinogen and thrombin (6) allows uniform mixing of the solutions so as to provide the polymerization mixture. The mixing device (6) is connected with the container for the fibrinogen solution (1-2) and the container for said thrombin solution (1-4) by ducts (3-4, 3-5) wherein the solutions are transported from the containers to the mixing device. In FIGS. 1 to 3 the duct transporting the polymerizing mixture of fibrinogen and thrombin and the duct transporting the separation medium are connected by a′ T-shaped connector (4). FIG. 3 shows an embodiment of the invention with a hollow tool (7) that will fulfil two functions: The first one to be used as a tube in which the fibrin segments will be formed while being able to be adapted on one side to the application system and on the other side with the design of spine fusion cages (8). The surgeon can use such an assembly to produce and deliver the fibrin segment either partially or fully polymerized into the cage (8) prior to position it in between the inter vertebra bodies or the opposite.

The performance of the method according to the present invention may be exemplified by FIGS. 1 to 3: The polymerization mixture (9) and the separation medium (S) can be applied to the device using the inlets (1) and (2). The flow of mixture and medium are conducted in ducts (3-1, 3-2, 3-3) for mixture and medium. During transport of the polymerization mixture in the ducts, polymerization reaction is performed. A continuous transport (flow) is enabled by external forces (e.g. gravity, pressure, etc.) into the direction indicated by arrows in the figures. The continuous flow of the polymerization mixture is interrupted with the separation medium (4) thereby obtaining consecutive volumes of the polymerization mixture and volumes of separation medium (see the S/P/S/P . . . in FIGS. 1 and 3). These consecutive S/P/S/P . . . volumes are further transported wherein the polymerization mixture further polymerizes and a discontinuous polymerized product is obtained, which can then be removed (e.g. at the right efflux of the device shown in FIGS. 1 and 2 (e.g. into a container or into storage ducts or tubes) or into the spine fusion cage of FIG. 3).

Fibrinogen and thrombin are perfectly mixed using the fibrin polymerization device according to the present example for the invention (herein also referred to as “Inline Mixing Technology”. Then air is added to fibrin at defined flow ratio to form a flow constituted of fibrin pearls separated by air segments.

This process can be conducted in a container having a constant section such as a plastic tube, catheter, tool as spine fusion cage tool or holder. In the present experiments, the specific ducts mentioned below are applied. This process can be scientifically named “Air flow segmented Fibrin” leading to the formation of a fibrin necklace or fibrin pearls if connected or not.

According to the present experiment, fibrinogen and thrombin are mixed through a mixing device (MIX-U) (MIX-U is a mixing device containing a single disc, MIX-C which contains two VYON F discs (Porvair, UK) could also be used) and polymerization takes place into tubing for 30 min. Air is introduced at equal flow rate of 2 ml/min set up on the Harvard pump which means a total of 4 ml/min into the plastic tube (diameter 1.4 mm). MIX-U is connected before the T shape connector to ensure a good mixing of fibrinogen and thrombin (6 in FIGS. 2 and 3). The Inline Mixing Technology is described e.g. in EP1973475A.

In a prototype of the device according to the present invention', a Duploject® device is provided (as embodiments of 1-1,1-2, 1-3, 1-4 and 5 of FIGS. 2 and 3) which is equipped with two 5 ml syringes that are filled with air. One contains 5 ml of fibrinogen and the other 5 mL of thrombin 4 IU/ml. The syringes from each Duploject® are connected with Y pieces. A Mix-U device containing a VYON-F disc is connected to the Y piece to ensure that fibrinogen and thrombin will be properly mixed.

Silicone tubes are used to connect the outlet of the Mix-U and the other Y piece via a T shape connector. The exit of the connector is connected to a tubing. In this device the two fibrin glue components are perfectly mixed together before the addition of air. Then air can clearly segment the fibrin flow in a sequence that can be controlled by monitoring the fluid mechanic parameters. FIG. 4 illustrates three tubes containing Air Segmented fibrin materials. Fibrin segments are white while air segments are grey. The length of the fibrin segments can be controlled by increasing or decreasing the air flow, fibrinogen and thrombin flow as well as the tubing diameter.

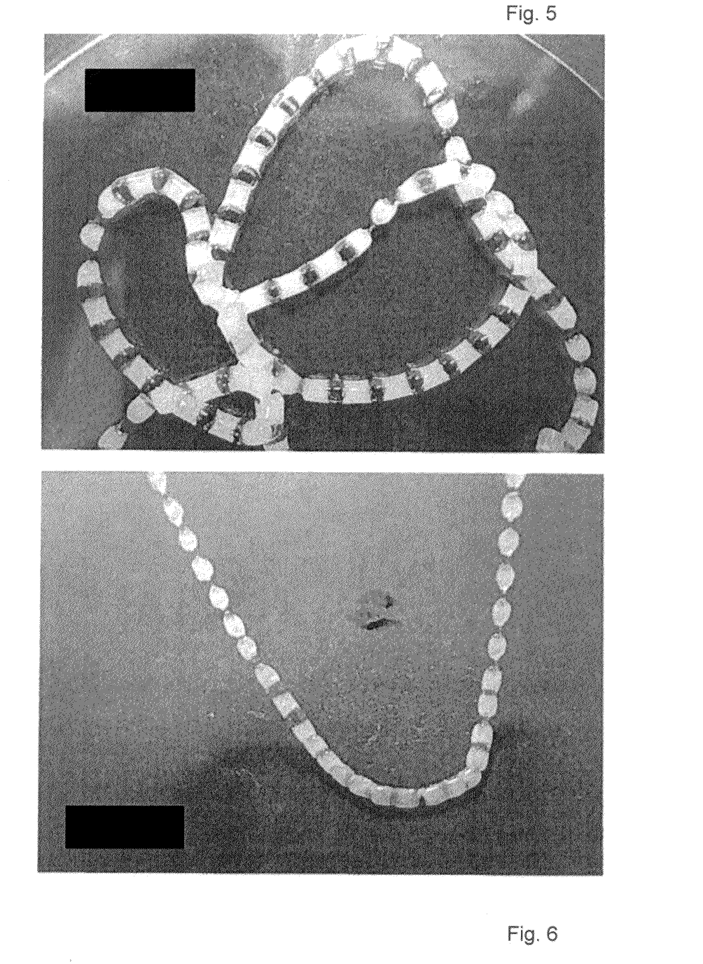

Time is no longer an issue as polymerization occurs in the tube from which it can be extruded at any time. The product can be ideally prepared early in the surgical procedure by the scrap nurse. A pre-polymerized segment for fibrin can be interconnected or free from each other by using tubing having appropriate surface tension. A regular PVC or silicone plastic tube will conduct to fibrin pearls that will remain connected by air bubbles (FIG. 5) while a Teflon coated tube will deliver fibrin pearls independent from each other (FIG. 6).

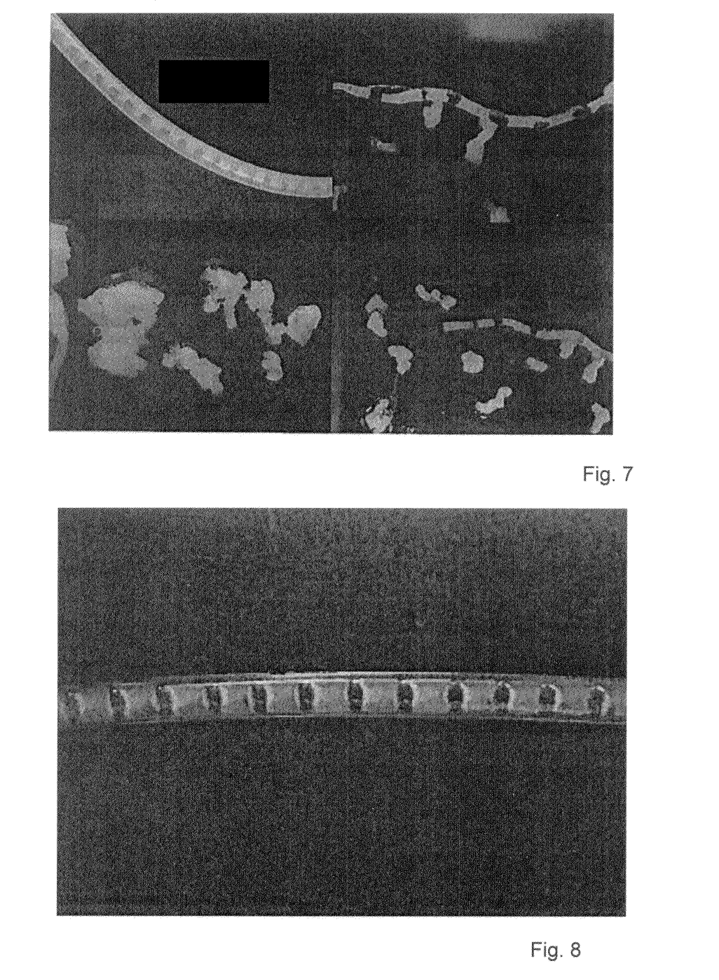

FIG. 7 shows the silicone tube (diameter 1.5 mm) containing the Air segmented Fibrin material (upper left). Upper and lower right illustrates that the fibrin material can be extruded linearly at low speed while forming clusters when fastly extruded. As a control, the same process was conducted but without MIX-U (FIG. 8): The control without mixing device tends to form a necklace with pearl distribution that is less reproducible, thrombin and fibrinogen are not well mixed and free thrombin is visible after extrusion (30 min waiting time).

With respect to fluid mechanics, bolus flow may be an appropriate concept for the present device and process: In many capillaries erythrocytes travel singly, separated by segments of plasma (bolus flow). The peculiar flow pattern, within the plasma, has been studied visually in a model in which air bubbles separated by short columns of liquid flow through a glass tube. Injection of dye reveals an “eddy-like” motion, in that each fluid element repeatedly describes a closed circuit. The possible significance of this “mixing motion” in relation to gaseous equilibration (e.g., in pulmonary capillaries) has been studied in a thermal analogue. A copper tube passed first through a constant temperature bath which brought the fluid to a uniform temperature T1, and then through a second smaller bath at a lower temperature T. From the final temperature T, of the fluid, which was collected in a thermally insulated flask, a calculation of the heat transfer was made (i.e. from the flow and the temperature drop (T1-T.)). Bolus flow was up to twice as effective in transferring heat as Poiseuille flow (no bubbles in fluid). The theory of modelling was employed in order to apply the thermal data to gaseous equilibration, especially in pulmonary capillaries. It was concluded that gaseous equilibration may be considerably accelerated by bolus flow, though this may be more of a limiting factor in peripheral capillaries than in the pulmonary circulation. The result supports the assumption of complete mixing in plasma.

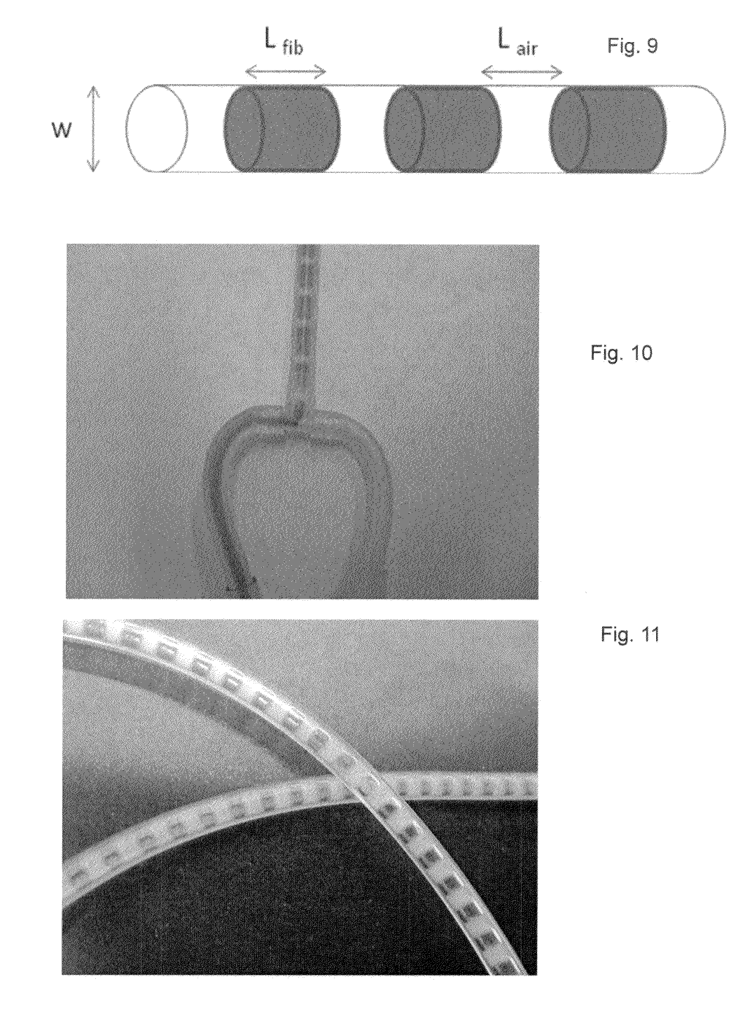

The scheme for the air flow segmentation is given in FIG. 9. “W” giving the internal diameter; “L_{fib}” the length of the polymerizing mixture volume segment; “Lₐᵢᵣ” the length of the separation volume segment.

### General Description of the Experimental Set-Up for Polymerization of Fibrin (See Also: FIGS. 1 to 3):

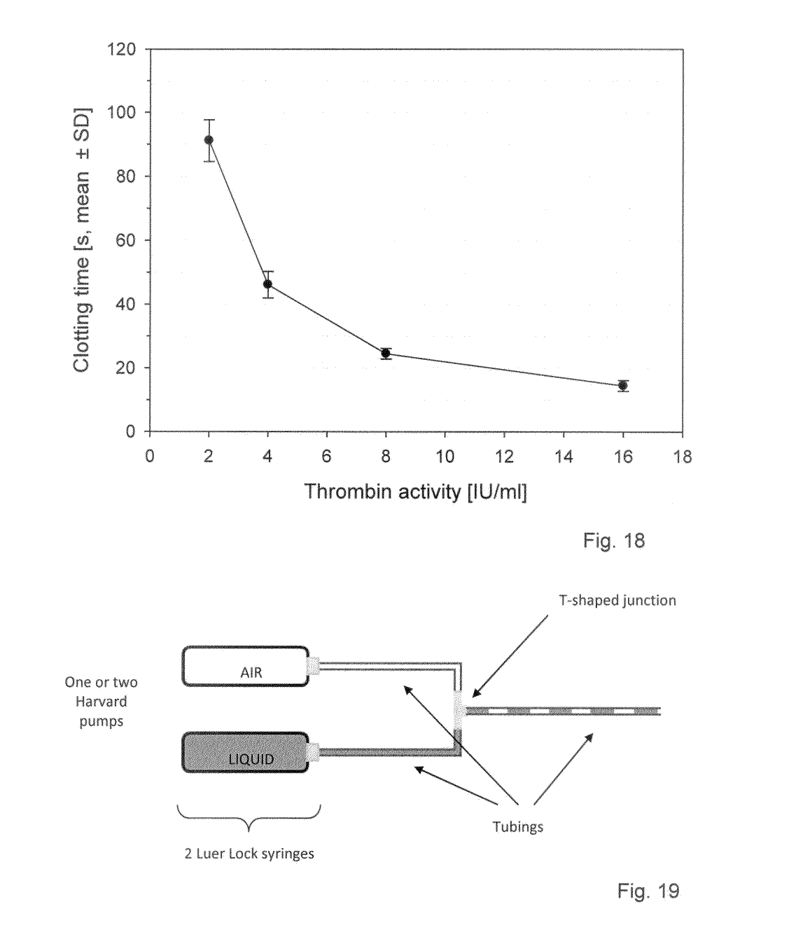

For tuning conveying fibrin (F) and separation medium (SM) having the same section, if flow of F is greater or lower that the flow of SM, the following can be expected. (of course, it has to be considered that the tube that can be adapted at the “T” junction may have different internal diameter thereby changing the dimension for the fibrin segments as flow is related to velocity of the liquids through a determined section tube): The advantage of a tube that is adapted is that it can be removed and fully implanted. The tube can be made of biodegradable. porous material. The tube containing the material that has been mixed in the polymerization device is depicted in FIGS. 1 to 3, basically all the tubes that contain the “material” (P) resulting of the mixing of fibrinogen and thrombin. Given the known information of the dependence of clotting time vs. thrombin activity, especially at low thrombin concentration (see FIG. 18), that an exponential increase of clotting times with decreasing thrombin concentrations is present, a person skilled in the art knows that clotting times between 40-50 s are realistic for a 4 IU/ml thrombin dilution.

Under the assumption of a cylinder formed by 1 mL of liquid (1000 mm³) in tube having different diameters, respectively 2 mm, 4 mm and 6 mm the following calculations may be drawn:

Calculation of the surface and length occupied by 1 cc in the liquid into the tubes

R_{tube}=1 mm

S_{segment}=3.14×1²=3.14 mm² S=section

L_{segment}=1000/3.14=318.5 mm L=length

R_{tube}=2 mm

S_{segment}=3.14×2²=12.56 mm²

L_{segment}=1000/12.56=79.61 mm

R_{tube}=3 mm

S_{segment}=3.14×3²=28.26 mm²

L_{segment}=1000/28.26=35.38 mm

Calculation of the lateral surface of fibrin segments formed in these tubes

R_{tube}=1 mm; SL_{segment}=2πr L=2×3.14×1×318.5=2000 mm²

R_{tube}=2 mm; SL_{segment}=2πr L=2×3.14×2×79.61=1000 mm²

R_{tube}=3 mm; SL_{segment}=2πr L=2×3.14×3×35.38=666 mm²

Calculation of the total surface of fibrin segments formed in these tubes

R_{tube}=1 mm; S=2(2πr)+SL_{segment}=2×(2×3.14×1)+2000=2006 mm²

R_{tube}=2mm; S_{segment}=2πr L=2×3.14×2+1000=1025 mm²

R_{tube}=3 mm; S_{segment}=2πr L=2×3.14×3+666 mm²=722 mm²

Ratio “Total Surface/Volume” of fibrin segments formed in these tubes

R_{tube}=1 mm S/V=2

R_{tube}=2 mm S/V=1

R_{tube}=3 mm S/V=0.7

It is evident that for a volume of 1 mL, the ratio S/V can be multiplied by a factor 2 or 3 when the diameter is divided by a factor 2 or 3. Increasing the surface will affect the pharmacokinetic as well as the residence time of the fibrin segment.

As a reference, a sphere having a volume of 1 ml, has a radius of 6.37 mm which correspond to a surface of 509 mm².

Assuming that the fibrin is applied as droplet (spherical) of 1 mL, the surface would be 509 mm², 5 times lower than a fibrin segment having a diameter of 1 mm.

A cube of 1 ml would have a surface of 600 mm².

For a cylindrical shape, the law that determine the ratio S/V is the following: S/V=2/R

R=1 S/V=2

R=2S/V=1

R=3 S/V=0.666

### Practical Experiments

### Experiment 1: Water/Air

Tubing: PVC, Internal diameter=1.4 mm; Total flow rate QT=4 ml/min; flow rate air=2 ml/min; flow rate water=2 ml/min

Lₐᵢᵣ: 0.875 mm; volume of air=1.4 μl
L_{water}: 2.45 mm; volume of air=3.77 μl

The result is depicted in FIG. 10.

### Experiment 2: Fibrin/Air

Tubing: PVC, Internal diameter=1.4 mm;
Total flow rate QT=4 ml/min;
flow rate air=2 ml/min; flow rate fibrin=2 ml/min
Lₐᵢᵣ,: 0.635 mm; volume of air=1 μl;
L_{fib}: 0.9144 mm; volume of fibrin=1.40 μl

The result is depicted in FIG. 11.

### Experiment 3: Fibrin/Air

Tubing: PVC, internal diameter=1.4 mm;
Total flow rate QT=5 ml/min;
flow rate air=2 ml/min; flow rate fibrin=3 ml/min
Lₐᵢᵣ: 0.70 mm; volume of air=1.1 μl;
L_{fib}: 1.36 mm; volume of air=2.1 μl

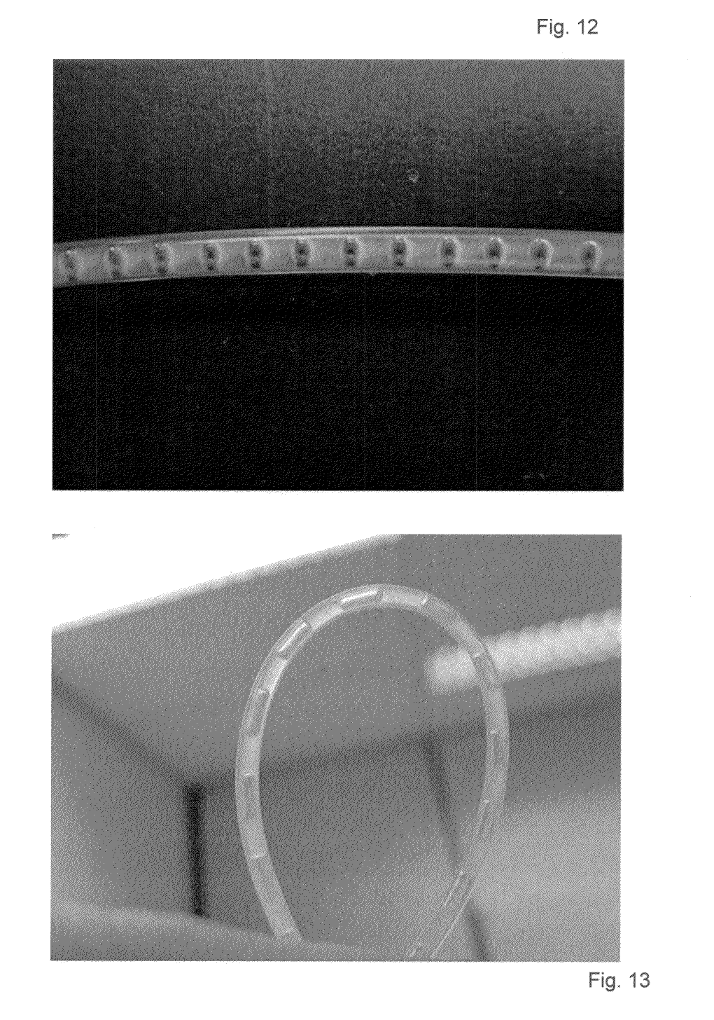

In this experiment, the fibrin flow rate was increased to 3 ml/min which increases the fibrin segment length. The result is depicted in FIG. 12.

### Experiment 4: Fibrin/Air

Tubing: Teflon; diameter=0.8 mm;
Total flow rate QT=4 ml/min;
flow rate air=2 ml/min; flow rate fibrin=2 ml/min
Lₐᵢᵣ: 2.56 mm; volume of air=1.28 μl
L_{fib}: 1.76 mm, volume of fibrin=0.88 μl

The result is depicted in FIG. 13.

From these experiments, the following conclusions can be drawn with respect to the optimization of the fibrin polymerization in the device according to the present invention: The main factors involved in the droplet/bubble formation and size are (see also: Tan et al., Chem. Eng. J. 146 (2009), 428-433):

The diameter of the channel (duct)

The respective flow rate of the gas and the liquid

The physical properties of the liquid phase (polymerizing mixture) and the nature of the tubing material (e.g. surface tension)

The respective angle between the channels (ducts), experience done above used the T-junction for the injection of liquid 1 and gas 1. The angle of injection of gas 1 channel could be lower than 90°.

A more complex design (e.g. Segudo et al., J. Flow Inj. Anal. 19 (2002), 3-8) can easily be applied with addition of a second liquid, so as to obtain a sequence of segments such as:

liq1-Liq2-gas1/liq1-Liq2-gas1/liq1-Liq2-gas1
The tube can be warmed up or cooled down during injection or any time after the injection.
The tube containing the fibrin pearls can be freeze dried.
The tube can be detached from the T shape connector, closed by sealing one or both sides, or with a plug. It can have a standard Luer on both extremities.

The principle on “Air segmented flow” is extensively disclosed in the present field (e.g. http://www.labautopedia.org/mw/index.php/Sample transport techno logy): One of the first automated laboratory systems to offer true high-throughput analysis was based on the principle of Continuous Flow Analysis (CFA) or Segmented Flow Analysis (SEA). This was the Autoanalyzer, invented 1957 by Leonard Skeggs, PhD and commercialized by Jack Whitehead's Technicon Corporation. The AutoAnalyzer profoundly changed the chemical analysis concept to a mindset that hundreds, or even thousands, of tests are possible per day. The autoanalyzer approach is described via the LUO concept as follows (LUO=A sequence of common laboratory steps or functions that when combined become a “unit” operation is referred to as a Laboratory Unit Operation (LUO)):

### Air Segmented Fluidic Flow

Sample Transport: A continuous, peristaltic pumped stream of liquid samples and reagents are transported and combined throughout the assay in Tygon tubing. Flows are in the range of millilitres/minute in tubing of approximately 2 mm diameter.

Sample Processing: The samples and reagents pass through the tubing from one sample processing device to another with each device performing different LUO's, such as mixing, distillation, separation (i.e. dialysis, extraction, ion exchange) and incubation.

Data Collection and Handling: The completed reaction mixture is pumped through a detection device (typically a UV detector) and subsequent a signal is recorded (strip chart recorder).

The Autoanalyzer architecture has proved to be very robust, with over 50,000 systems sold. These systems are relatively inexpensive, rugged and highly reconfigurable to accommodate different procedures. The 1970 technology update, Autoanalyzer II, can still be found in use today, running EPA reference methods that were created around the system. In 1974 a similar and commercially competitive technique, Flow Injection Analysis (FIA), was introduced. This technique was further refined and miniaturized, first via capillary flow techniques and eventually evolved to the current microfluidic technology through the use of semiconductor fabrication technology. Variations on the CFA technique continue to be developed.

### Experiment 5: Withdrawal Means for Separation Medium

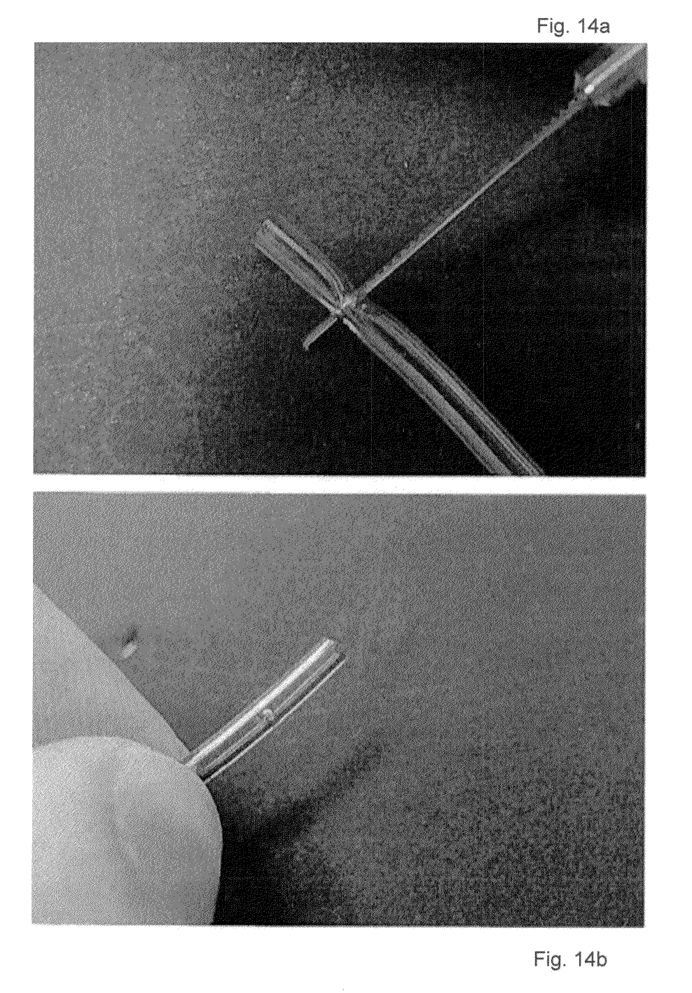

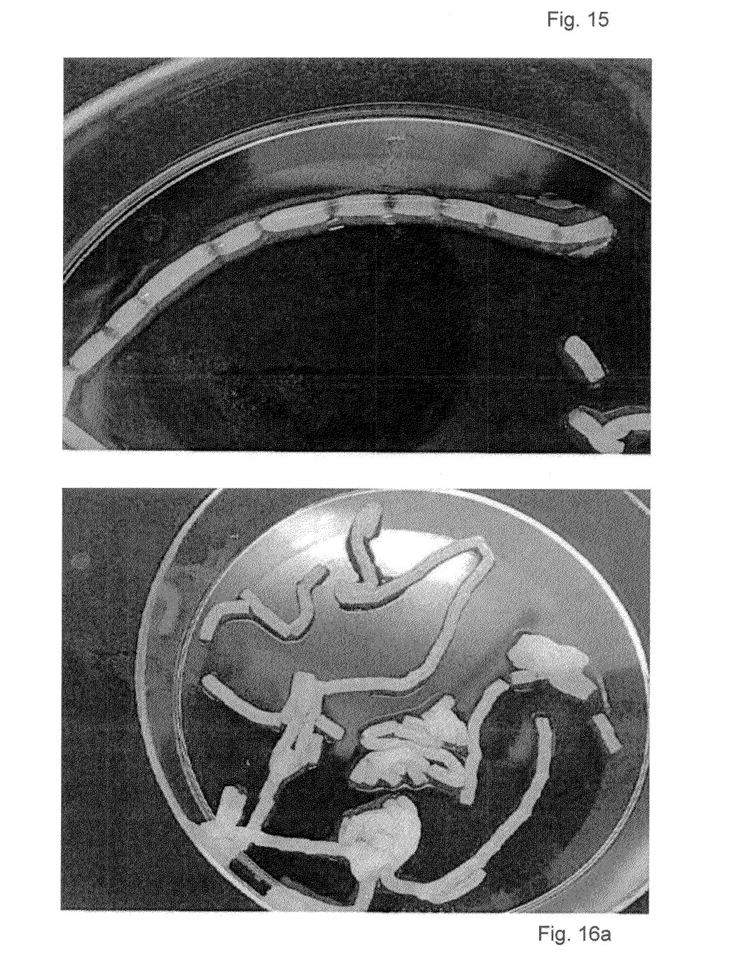

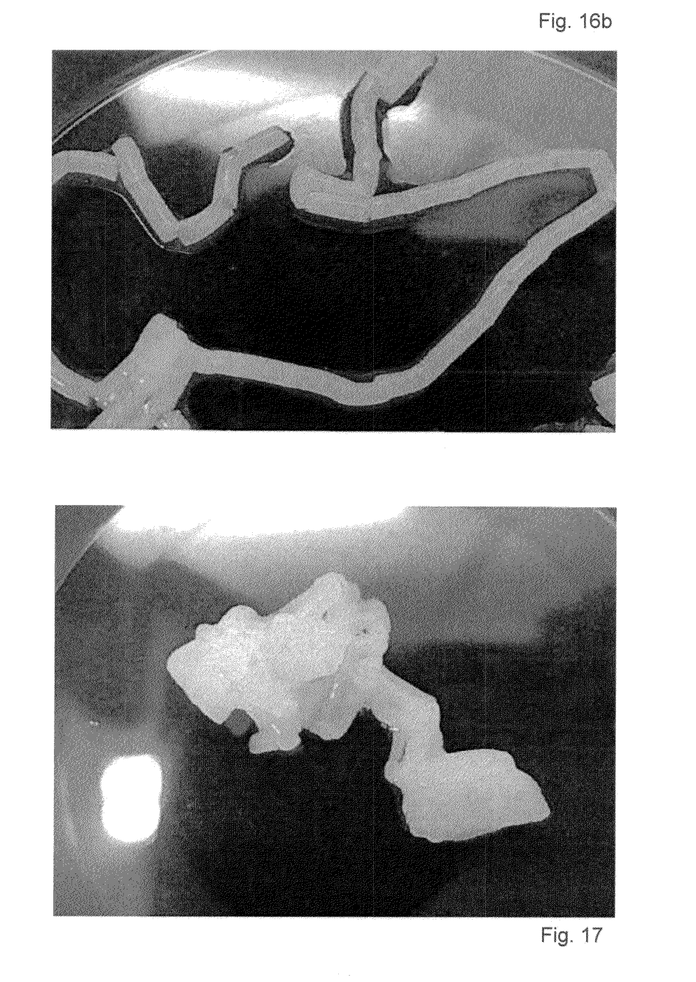

In this experiment, holes are provided as air vents (withdrawal means for the separation medium). FIGS. 14a and b show how the holes were made in the plastic tube using a metallic cannula with an unbeveled needle.

Tubing 1.4 mm diameter from extension set Baxter
Syringe 5 mL: Omnifix. B-Braun

### Syringe 20 mL: BD Plastipak

MIX-U 1 disc VYON-F:
Y piece from Baxter set device

Fibrinogen and thrombin are mixed through a mixing device, MIX-U and polymerization takes place into tubing for 30 min. Air is introduced at equal flow rate of 2 ml/min set up on the Harvard pump which means a total of 4 ml/min into the plastic tube (diameter 1.4 mm) with or without holes. Mix-U is connected before the T shape connector to ensure a good mixing of fibrinogen and thrombin. To demonstrate the effect of the separation medium withdrawing means, holes have been performed at the distal end of the plastic tube as shown in FIG. 14a and b). A control test was run with a tube without holes.

As shown in FIG. 15, the air segments in the control experiment show air bubble trapped into fibrin membrane. When making holes at the distal part of the tube, air is evacuated allowing the fibrin segments to pile up in a continuous succession of polymerized fibrin pearls (FIG. 16a and b). Once delivered, the polymerized fibrin pearls fold on themselves to occupy a minimum volume space as shown in FIG. 17. Such fibrin pearl structure free of air pocket can be used for filling the spine fusion cage. The air removal system is independent of the nature of the polymer/biopolymer that is conveyed into the tube and can be used for the Floseal and Coseal applications. The applicators can be rigid, soft, malleable or not. As mentioned, the device according to the present invention can be used to generate fibrin pearls. These can be directly connected to the holder used to handle the spine fusion cage. The holder shaft can be designed to have a hollow structure allowing the formation of polymerized fibrin pearls, internally, to directly end up inside of the spine fusion cage which is fixed at the end of the holder.

### Continuous or Discontinuous Fibrin Polymers:

### Materials

Tubing 1.4 mm diameter from extension set Baxter

### Syringe 5 mL: Omnifix B-Braun

### Syringe 20 mL: BD Plastipak

MIX-U with 1 disc VYON-F
Y piece from Baxter set device

### Method

Syringes are filled with fibrinogen (100 mg/ml) and thrombin at 4 IU/ml and placed on a Harvard pump. The syringes are connected to a “Y piece” which is connected to a mixing device type MIX-U.

The mixing unit is a piece of tube that is connected to a “T connector” on one side, the other sides of the T connector are respectively in communication with a tube that is conveying the separation medium and a tube in which segmented fibrin polymer will be stored.

The pump is actuated to deliver fibrinogen and thrombin in the ratio 1:1 at 2 ml/min for each syringe, so the final flow rate for fibrin is 4 ml/min

Fibrinogen and thrombin are mixed through a mixing device, MIX-U then segmented with air into a tube where polymerization takes place for 30 min.

Air is introduced at equal flow rate of 2 ml/min set up on the Harvard pump which means a total of 4 ml/min into the plastic tube (diameter 1.4 mm) with or without holes (to remove the membrane). Depending if the fibrin polymer has to be discontinuous or continuous, a tube equipped with flanges or not is used for carrying the biopolymer. Another option is to use the same tube and if a discontinuous polymer is required then the end tip device with pins is adapted at the distal part of the tube before the actuation of the device.

### Experimental Study Testing the “Squeezing” Regime Set-Up

In a specific set-up, the theoretical considerations for the “squeezing” regime (capillary number below about 0.01) in a polymerization device using a T-shaped junction are experimentally tested. The segmentation device for these tests is shown in FIG. 19, wherein silicone ducts (“tubings”) with inner diameters of 1, 1.5, 2 and 3 mm were used.

Key variables of such a system include the type of fluids, the length of the ducts and their inner diameter, the segmentation, the flow rate, the flow rate ratio and the time of the process.

### a) Type of Fluids

In a system wherein fluid 1=air and fluid 2=water, the following parameters would apply (in the present model, glycerol was used at same viscosity (85%) than fibrinogen to show the proof of concept (glycerol and water turned out to be a perfect model system for fibrinogen and thrombin, specifically with respect to viscosity properties). Also a final mix of glycerol 85% (which perfectly mixed with water) was used to show that the settings remain correct and applicable); moreover, “glycerol (50%)+water (50)” allowed to roughly reach the operational conditions that are fine-tuned later with the final product, in this case fibrinogen and thrombin):

| | Viscosity at 20° C. | Surface tension with |
|---|---|---|
| | (10⁻³ Pa · s = 1 cP) | air (mN · m⁻¹) |
| Water | 1 | 72 |
| Glycerol (85%) | 110 | 63 |
| Glycerol (50%) + | 5 | 70 |
| water (50%) | | |

* physical characteristics measured in using known standard methods

### b) Segmentation Mode

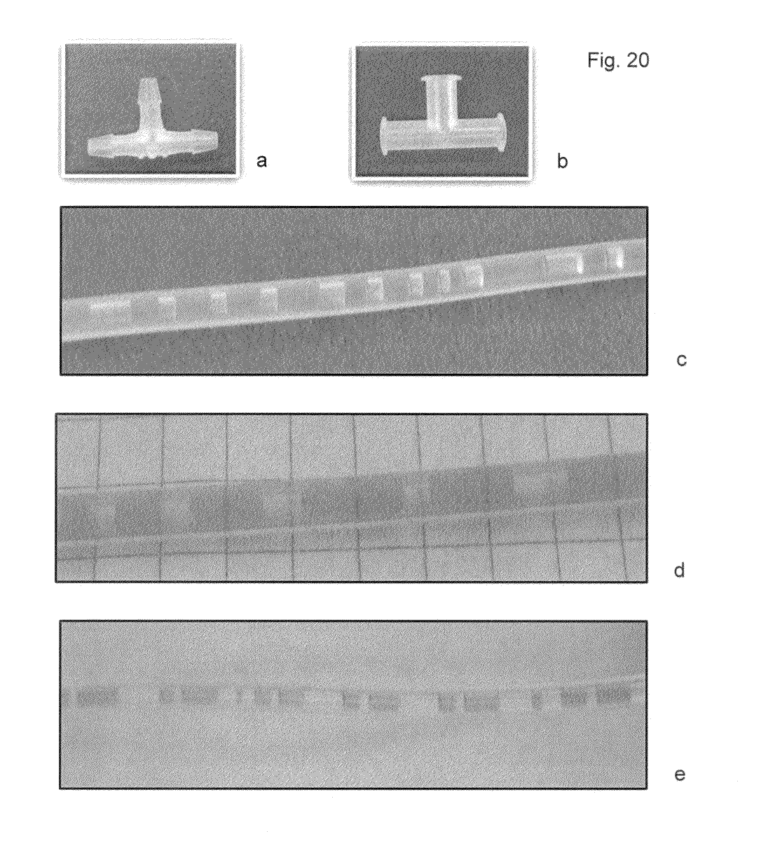

In a first set-up, Biorad™ junctions according to FIG. 20a (Junction Biorad 1 (JBr1); internal diameter: 1,20 mm) and 20b (Junction Biorad 2 (JBr2); internal diameter: 4 mm) are used. The water segmentation with these junctions are displayed in FIGS. 20c, 20d and 20e. Although segmentation is possible in principle, it is evident that the resulting product is not regular and the process is difficult to control.

It is known from studies concerning mandatory conditions to air segment a liquid flow that at low values of capillary number, when the interfacial forces dominate the shear stress, the dynamics of break-up of the immiscible fluid in T-junctions is dominated by the pressure drop across the droplet or bubble as it forms (Garstecki et al., Lab Chip 6 (2006), 437-446). Practically, in this squeezing regime the process of break-up is dependent on the flow rate and geometries. The size of the droplets or bubbles is determined by the ratio of the volumetric rates of flow of the two immiscible fluids.

FIGS. 20c, 20d and 20e show the segmentations results for water and methylene blue with air using the JBr1 junctions at different flow rates (1,25 ml/min, 3 ml/min and 0,50 ml/min). From these figures it is evident that although the process according to the present invention, including the air segmentation of a liquid flow, can be carried out with these T-shaped junctions in principle, the resulting product is not regularly shaped. This means that generation of air segment is not stable overtime, it cannot be controlled and therefore lengths of air and liquid segments are changing over time. This is due to the design and inner diameter of the junctions according to FIGS. 20a and 20b; preferred designs for the inner diameters for the ducts according to the present invention are from 0.005 mm to 5 mm.

In a preferred embodiment of the present invention, the liquid segments generated should have a constant ratio surface/volume. As tube has a constant diameter, by controlling length of the segment, the segment volume is controlled as well (Garstecki et al., 2006).

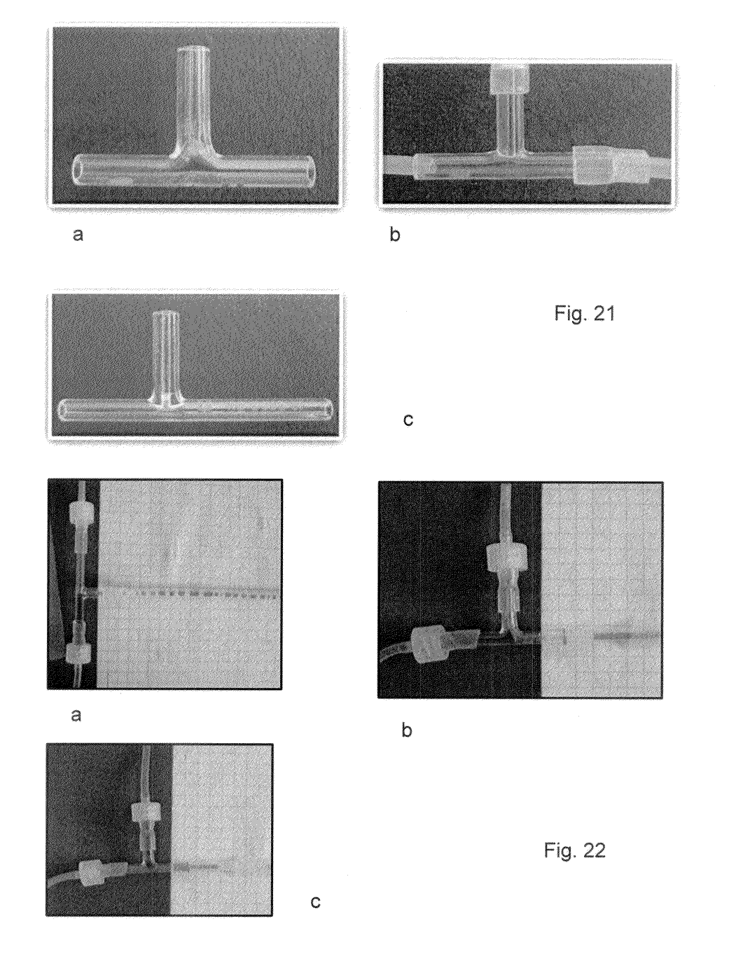

Accordingly, the other junctions were tested (see FIGS. 21a, 21b and 21c: FIG. 21a: junction Technicon™ 1 (“JT1”), 21b: junction Technicon™ 1 bis(“JT1bis) and 21c: junction Technicon™ (“JT2″). These junctions have preferred properties and are therefore preferred embodiments according to the present invention. More generally, such preferred embodiments can be defined as follows:
the outer diameter does not matter;
the length of the lateral and vertical branches do not matter as long as the T junction where the bubble is generated and growing is long enough;
the material can be glass, metal, polymer, mix thereof of basically any material that can be sintered;
the material has to resist to oxidative and reducing environment, high and low temperature, and the like; specific examples for such material are Titanium Alloy 20, Inconel 60, stainless steel 316L SS, etc., hasteloy X, hasteloy C-276, etc.

With these junctions, it was possible to create regular segments of glycerol, and results were reproducible with junctions JT1, JT1bis and JT2. Lateral entry internal diameter equals vertical entry internal diameter for the Bio Rad junction shown FIG. 20a and Bio Rad junction 2 shown FIG. 20b are respectively 1.2 mm and 4 mm.

Lateral entry internal diameter equals vertical entry internal diameter for the Bio Rad junction 1 and 2.

| | JT1 | JT1bis | JT2 |
|---|---|---|---|
| Lateral entry | 2.40 | 2.40 | 1.90 |
| intern | | | |
| diameter (mm) ± | | | |
| 0.05 mm | | | |
| Vertical entry | 1 | 1 | 1.1 |
| intern | | | |
| diameter (mm) ± | | | |
| 0.05 mm | | | |

Accordingly, for all following studies, the junctions according to FIGS. 21a, 21b and 21c (JT1, JT1bis and JT2 were used).

### c) Connection Mode

There are several ways of connecting tubes to the T-shaped junction in an air/liquid system: the air and liquid-are directed via the arms of the “T” and the product exits' via the stem of the “T”; the liquid enters from an arm, the air from the stem and the product exits via an arm; or the air enters from an arm, the liquid from the stem and the product exits via an arm (see FIGS. 22a, 22b and 22c). In the experimental set-up, it appeared directly that the first configuration was more likely to be disrupted. Indeed, in this configuration, a balance between both flows is really crucial in order to observe a regular segmentation. Thus, the slightest perturbation could break the stability and it seemed more difficult to control the phenomenon. On the contrary, the two other configurations gave similar results and a good stability.

Under consideration of a “squeezing” regime (capillary number (Cₐ=μ_{c}·ν_{c}/γ; (ν_{c}=dynamic viscosity; γ=surface tension)) inferior to 0,01 approximately), breakup of air bubbles occurs just at the angle between the main channel, and the air inlet. If working in dripping regime, this bubbles formation would have occurred downstream the junction Therefore, for one given liquid (it sets viscosity and surface tension) and one given junction (it sets geometrical dimensions), it will give the maximum flow rate it is possible to work at.

| | | | Maximum | |
|---|---|---|---|---|
| | | | flow rate | Maximum |
| | | Surface | with JT1 | flow rate |
| | Viscosity | tension | (or JT1bis) | with JT2 |
| | (Pa · s) | (N · m⁻¹) | (mL/min) | (mL/min) |
| Water | 10⁻³ | 73 | 200 | 130 |
| Glycerol | 0.113 | 63 | 1.6 | 1 |
| (85%) | | | | |
| Glycerol | 5.10⁻³ | 70 | 39 | 24 |
| (50%) | | | | |

### d) Time

Timing turned out to be easily manageable in the method according to the present invention; at least after some adjusting time after the pumps have been switched on, the system was stable.

e) Ratio of flow rates: Q=Qa/Qg (a=air, g=glycerol)

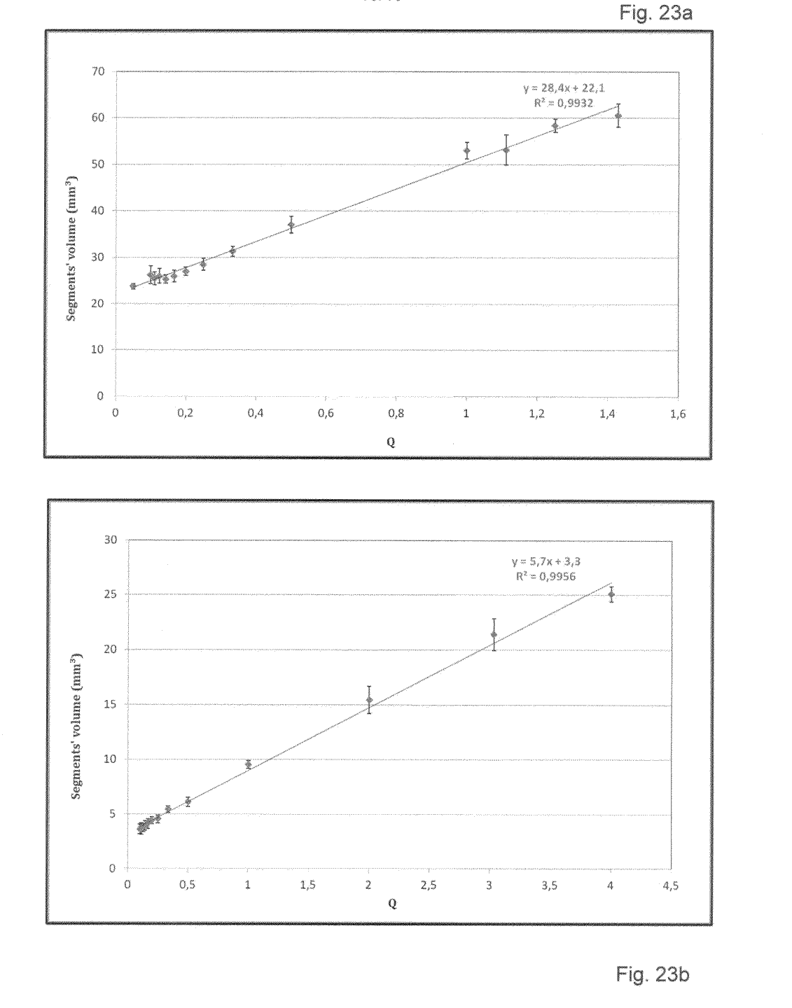

Here the influence of the ratio of flow rates was studied. The dependency of the segments' volume depending on the ratio of flow rate Q was analysed with JT1 (Q_{g}=0.1 mL/min, Q from 0.1 to 1.5, n=4; FIG. 23a) and JT2 (Q_{g}=0.1 mL/min, Q from 0.1 to 1.5, n=4; FIG. 23b). From the theoretical considerations, for a given couple of fluid, a given junction and a given type of tube, a linear relation between segments' size and the ratio of flow rates Q was expected that. In the range of flow rates chosen, results obtained are confirming this law. For both junctions, the correlation coefficient for a linear law is superior to 0,99 and the results are reproducible (n=4). Moreover, the maximum error was of 6%.

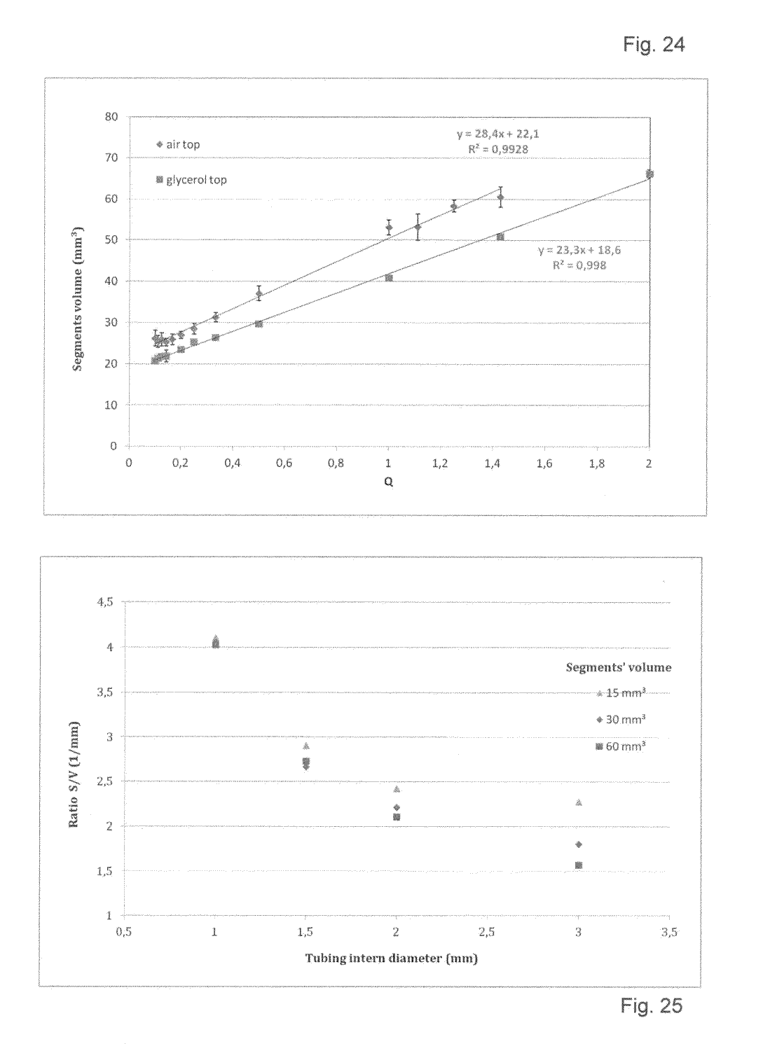

With respect to the injection mode for interchanging liquid and air introduction, two alternatives were tested (air from the stem (blue diamonds in FIG. 24 (“air top”)) and glycerol from the stem (orange squares in FIG. 24 (“glycerol top”))). The results are shown in FIG. 24 for JT1. Segments volume follows the same scale law for both configurations, it must therefore be the same process of break up.

f) Tubing length/intern diameter

Pressure is a key parameter so the internal diameter or length of the ducts (“tubing”) is also an important parameter.

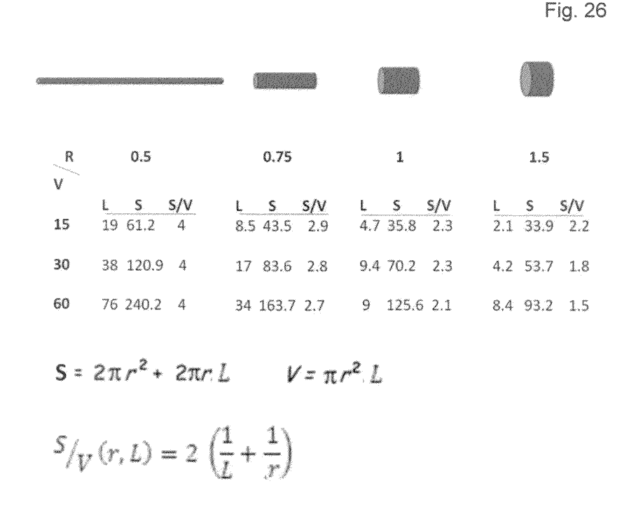

For a gas/liquid segmentation process, a linear relation is expected between the flow rate ratio Q and the volume (see FIG. 25 and 26). The tube size is also as it enables to obtain different ratios surface/volume with one configuration of settings. This ratio surface/volume is also a key parameter to study the release of a substance trapped e.g. in the fibrin network.

### Fibrin Segmentation

For these experiments, the experimental device and set-up was the same as described above. However, the liquid which is segmented by air is a mix. Raw products are fibrinogen (with methylene blue as coloring agent) and thrombin from TISSEEL kit which have to be mixed prior to segmentation. A Duploject system was applied which can be completed with a mixing device upstream the T-junction.

Under the “squeezing” regime of segmentation, the viscosities in the following table are calculated for different components, as well as the corresponding capillary numbers for mixes.

| | | Estimated capillary |
|---|---|---|
| | | number (Q = 4 mL/min, |
| | | tubing diameter = |
| | Viscosity | 2 mm, surface |
| | (at 130 s⁻¹, cP) | tension = 63 mN/m) |
| Fibrinogen 1:1 | 110.5 | |
| (undiluted) | | |
| Fibrinogen buffer | 1.6 | |
| Fibrinogen 1:2 | 6.4 | |
| (diluted with water) | | |
| Thrombin (500 IU/mL) | 1.5 | |
| CaCl₂ (40 μmol/ml) | 1.3 | |
| Thrombin (4 IU/mL) | 1.3 | |
| Fibrinogen 1:1 + | 6.0 | 0.002 |
| Thrombin (4 IU/mL) | | |
| Fibrinogen 1:2 + | 2.8 | 0.001 |
| Thrombin (500 IU/mL) | | |

### a) Mixing Quality

Provision of a mixing device for the components of the polymerization mixture is a preferred embodiment of the present invention. This advantageous feature is specifically used in the production of segmented fibrin polymers.

The following experiments were again worked under the squeezing regime. The mixing quality was important for an excellent performance of the segmentation process.

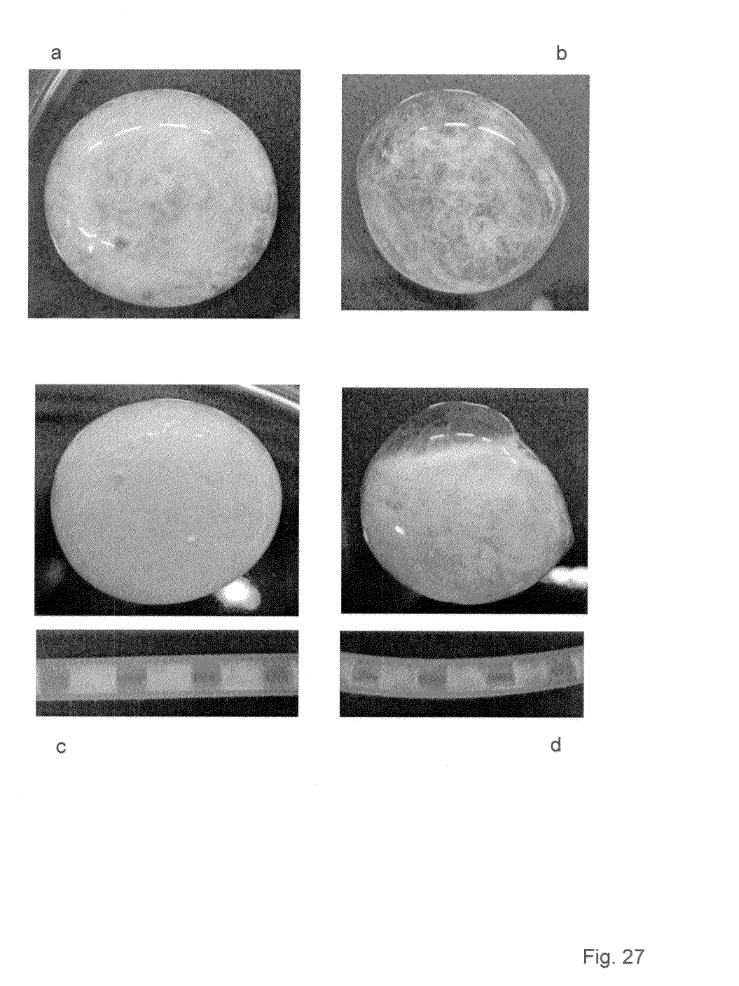

The experiments were performed with fibrinogen 1:1 (not diluted); thiombin 4 IU/mL; Q_{f+t} (flow rate fibrinogen and thrombin)=4 mL/min either with or without use of a Mix-C device, a mixing device with a porous disc (EP 2213245A; “device with 2 discs”; “device with 1 disc”). The resulting polymerization products are shown in FIG. 27.

FIG. 27a and FIG. 27b show the polymer after 30 s of polymerization (Duploject equipped with two syringes respectively filled out with fibrinogen 100% and thrombin at 4 IU/ml, is set up on an Harvard medical pump, flow rate is 4 ml/min for generating the droplets and when using the T junction and the tube). Fibrin droplet obtained with the MIX-C already appears to be more homogeneous that fibrin droplet obtained without mixing device after 30 s of polymerization.

FIG. 27c and FIG. 27d show the polymer after 5 min of polymerization and in the duct after the segmentation process. Fibrin droplet obtained with the mixing device is well polymerized, very homogeneous, single phase. This means that when the T junction is used for air segmentation of such an efficiently mixed fibrin, it can be expected to obtain well segmented fibrin in the tube as shown in FIG. 27c. Fibrin and air segments have similar length and are nicely cut. SEM pictures of cross sectional and longitudinal segment are confirming the homogeneity of the fibrin clot obtained when using the MIX-C before segmentation. On the other hand, fibrin obtained without using a mixing device is not fully polymerized, pure unreacted thrombin and fibrinogen are remaining which give transparent areas within the fibrin clot and on the top of the droplet. This multiple phase liquid is not favorable to facilitate an easy break-up or segmentation by air leading to air and fibrin segment that have irregular lengths as shown in FIG. 27d.

It is therefore evident that fibrin products which come from a mixing device with a porous disc (FIG. 27a and FIG. 27c) are superior compared to a mixing device without any porous disc (FIG. 27b and FIG. 27d, upper figure). It can be seen that a mixing device with a porous disc (e.g. a Mix-C device) provides a far better polymer. This observation was also confirmed with segments of fibrin (FIG. 27b and FIG. 27d, lower figure).

It follows that excellent air segmentation of fibrin can only be obtained if mixing of the two fibrin glue components is at optimum. Segmentation cannot be regular and stable over time if the fluid coming at the T-junction is a mix of fibrin/pure thrombin and fibrinogen. An efficient mixing device, MIX-C in this case (MIX-U; EP 2 213 245 A) or similar devices can be used too), is used for mixing fibrinogen full strength and thrombin 4 IU/ml.

In another embodiment the mixing device contains at least one disc placed after the T junction to foam the fibrin segments with the air segment so that foamy segmented fibrin may be obtained.

### b) Kinetic of Polymerization

In this fibrin polymerization model, the kinetic is mainly influenced by both thrombin and fibrinogen concentrations. Flow rate has to be adjusted to avoid polymerization in the equipment; it is therefore preferred to use a high flow rate and to have low concentrations.

In the present experimental conditions, a thrombin concentration lower than 10 IU/mL was used; the fibrinogen concentration was adapted with a dilution factor from 1 to 4.

As junction JT2 and JT1bis were used. It was observed that with junction T1 (due to the triangular shaped space where air and liquid gather) fibrin accumulation was “promoted” and junction blocking may occur.

### c) Extrusion of Segments

In the present experimental set-up, Teflon tubes were used which are less sticky than silicone tubes. With a thrombin concentration of 4 IU/mL; whatever the fibrinogen concentration, fibrin was allowed to polymerize at least 30 min in order to be able to extrude segments.

Extrusion was mainly dependent on the fibrinogen concentration and thrombin concentration. During the extrusion process, the application of pressure tended to separate the aqueous phase from the polymerized one so it was decided that fibrinogen was not diluted at all in order to minimize the amount of aqueous phase in the segments. Extrusion was performed by pushing the segments with air.

### d) Experimental Set-Up

Due to these considerations, the following experimental parameters were set for the further studies:

| Parameters | Setting |
|---|---|
| Junctions | T1bis and T2 |
| Fibrinogen concentration | Non diluted (“1:1”) |
| Thrombin concentration | 4 IU/mL |
| Fibrin flow rate | From 2 to 8 mL/min |
| Mixing device | Mix-C |
| Extrusion | With air, after a whole night of |
| | polymerization |

### Pharmacokinetic Study

As shown above, it was possible to produce segments of fibrin with different ratios surface/volume and with different thrombin concentrations in a process and device according to the present invention. The next step was to study how these parameters might influence the pharmacokinetic profile of a substance in vitro.

In the present experimental set-up a substance was added to fibrin segments which would be easy to follow; then, to put segments in a solvent during several hours to study how the substance would release out of them. Therefore, a system with was chosen. Fibrin is a network of polymer filled with an aqueous phase. Methylene blue (MB) and doxorubicin (DX) are trapped inside the network and their release from the network by a diffusion process can be studied.

This release is based on the diffusion of a molecule out of the fibrin network. Considering that molecules size is around a nanometer and the order of magnitude of a pore size in the fibrin network is around a micrometer. Therefore, whatever the pore size, the molecule release might not be influenced by the fibrin network structure.

For the present experiments 4 different samples of fibrin were prepared:

| | | | | Fibrinogen + | | | |
|---|---|---|---|---|---|---|---|
| | | | | thrombin | Air | Volume of | Ratio |
| | Thrombin | | MB or DX | flow rate | flow rate | segments | surface/ |
| | conc. | Fibrinogen | conc. | (mL/min) | (mL/min) | (mm³) | volume |
| Sample 1: | 4 IU/mL | Non | 0.5 mL of a | 4 | No | Cube of around | 0.7 |
| ref. | | diluted | MB solution | | segmentation | 1000 mm³ | |
| Sample 2 | | (just | at 1 mg/mL | 4 | 2 | 23 mm³ | 2 |
| Sample 3 | | addition | in 5 mL of | 4 | 5 | 7 mm³ | 3 |
| Sample 4 | | of MB) | fibrinogen | 4 | 5 | 7 mm³ | 4 |

For each sample, the protocol was the same:

Perform segmentation during several seconds until stabilization;

remove the tube from the T-junction before stopping the pump because tubes were refrigerated one night long in order to achieve correctly the polymerization;

Extrude segments

With this method, it was easy to compare the results for each sample in order to analyze the influence of the ratio surface/volume.

### a) Methylene Blue Release

1. Presentation and properties

Methylene blue (MB) is a molecule which is commonly known as a dye. Indeed, when dissolved in water, it gives a blue solution. It is often used as a simple dye, for example in food industry, but also as a redox or pH indicator in many chemical reactions.

### 2. Results

Four different samples were prepared as described above having different ratio surface/volume. A known quantity of water was added; this mixture was preserved them in falcons; the samples are kept at room temperature between measurements (“experiment 1”).

The results obtained are disclosed in FIG. 28a. It can be observed that the amount of MB which is present in water evolves over the time: during the six first hours, MB is released in the solvent, and after, a sharp diminution of MB concentration in surrounding water was observed. The higher the ratio surface/volume, the faster the release from the segments.

| | 30 min | 3 h | 6 h 30 |
|---|---|---|---|
| Sample 1 | 20% | 32% | 45% |
| Sample 2 | 31% | 45% | 50% |
| Sample 3 | 34% | 50% | 52% |
| Sample 4 | 48% | 57% | 55% |

Indeed, sample 4 reaches quasi instantly its maximum value of release, whereas it takes around 24 hours to sample 1 to reach it. Differences between sample 2 and sample 3 are more difficult to affirm taking into account errors bars, but it is sure that they both release faster than sample 1 and slower than sample 4 (see FIG. 28b). It was quite easy to forecast the fact that MB concentration in water increases with the time, whereas the decrease from around 6h after the beginning is quite surprising. Indeed, a drop of MB concentration in the solvent was observed. These results are repeatable because this experiment was performed twice, with exactly the same protocol.

In a further experiment, the samples are kept refrigerated between measurements (“experiment 2”), however, the same kind of profile was observed (see FIGS. 29a and 29b). This confirms the dependence on the ratio surface/volume and, for the beginning, the dependence on time. On the other hand, one can see that the drop of concentration, after several days, is not avoided but is smaller than with “experiment 1”.

### 3. Discussion

The dependence on ratio surface/volume is easily understandable taking into account the fact that the bigger the surface, the higher the quantity of MB which has a really little distance to diffuse on. Indeed, diffusion time decreases with the distance. Thus, the bigger the ratio surface/volume, the fastest the diffusion process is. This point is of great interest for surgical applications. Indeed, as one just has to adjust flow rates in order to choose the ratio surface/volume of fibrin segments, it is easy to choose which speed of release is desired and to adapt the experimental parameters in order to create the corresponding segments.

### b) Doxorubicin Release

1. Presentation and properties

Doxorubicin (DX) is a molecule often used as a drug for the treatment of cancers, particularly leukemia. It works by intercalating DNA. It is presented as a hydrochloride salt and is red. It is a photosensitive product which must, consequently, be kept in a dark place. It is easily dissolved in several kinds of solvents as water, ethanol, etc.

Fibrin does not have any influence on DX, and DX living time was higher than 14 days. DX has some fluorescence properties which can easily be used to measure its concentration in a solution. Indeed, excited with a 470 nm light, it emits a 593 light which intensity depends on DX concentration.

### 2. Results

Exactly the same protocol as above for MB release was used for this second set of experiments.

Experiment carried out twice. DX has to be kept at low temperature and moreover, it is photosensitive, that is why samples were kept refrigerated between experiments.

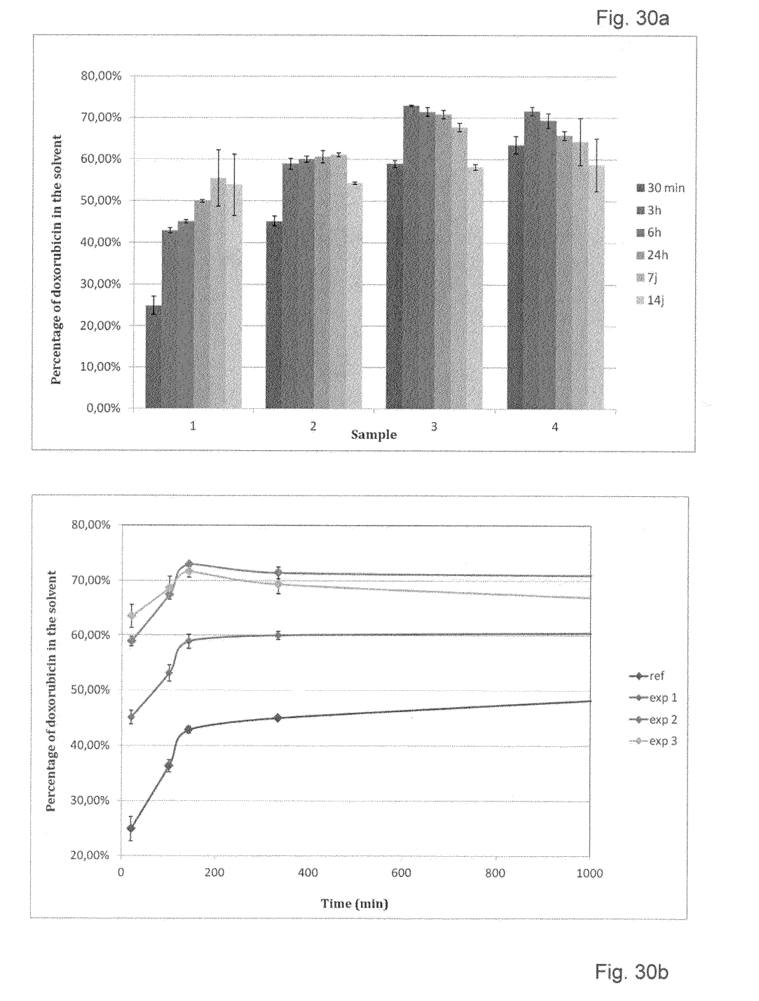

The results are depicted in FIGS. 30a and 30b. The same trend as in the MB release experiments was observed depending on the ratio surface/volume.

Preferred embodiments of the present invention can be defined as follows:
1. Method for the production of a polymerized product comprising the following steps:
providing a polymerization device to which a polymerization mixture and a separation medium can be applied and wherein flow of said mixture and medium can be conducted in appropriate ducts for said mixture and medium,
transporting said polymerization mixture in a duct of said polymerization device thereby allowing the polymerization reaction,
transporting said mixture in a duct of said polymerization device in a continuous flow,
interrupting said continuous flow of said mixture with said separation medium so as to obtain consecutive volumes of said mixture and volumes of said separation medium,
further transporting said consecutive volumes of said mixture and volumes of said separation medium in a duct of said polymerization device wherein said mixture further polymerizes to obtain a discontinuous polymerized product, and
removing said discontinuous polymerized product from said polymerization device.
   2. Method according to embodiment 1, wherein said polymerization mixture is selected from a mixture of fibrinogen and thrombin, a mixture of gelatine and thrombin, a mixture of polysaccharide, especially alginate, and calcium, a mixture of polysaccharide and isocyanate, a mixture of poly(vinyl alcohol)-alginate and calcium, a mixture of albumin and aldehyde, a mixture of chitosan and glutaric dialdehyde, a mixture of chitosan and glycerol-phosphate disodium salt, a mixture of collagen and glutaraldehyde, a mixture of gelatin and glutaraldehyde, a mixture of polyethyleneglycol and amino acid with reactive end groups, a mixture of alginate—polyethyleneglycol diamines and carbodiimide.
   3. Method according to embodiment 1, wherein said polymerization device comprises at least one pressuring device for transporting mixture and medium, said pressuring device is preferably a pump or a plunger.
   4. Method according to any one of embodiments 1 to 3, wherein said polymerization device comprises at least two containers for components of said polymerization mixture, said mixture being composed of at least two components.
   5. Method according to any one of embodiments 1 to 4, wherein said polymerization device comprises a mixing device for said components so as to obtain said polymerization mixture.
   6. Method according to embodiment 5 wherein said mixing device is selected from the group consisting of a Y-shaped connector, a filter material, a three-dimensional lattice or matrix material.
   7. Method according to embodiment 5 or 6 wherein said mixing device is connected with said containers by ducts wherein said components can be transported from said containers to said mixing device.
   8. Polymerization device suitable for carrying out the method according to any one of embodiments 1 to 7.
   9. Polymerization device according to embodiment 8, wherein the polymer mixture contains components selected from the group consisting of a biopolymer precursor, especially fibrinogen, thrombin, collagen, alginate, chitosan and mixtures thereof.
   10. Polymerization device according to embodiment 8 or 9, wherein at least one duct in said polymerization device contains withdrawal means for said separation medium to withdraw said separation medium.
   11. Method for the production of a fibrin product comprising the following steps:
providing a fibrinogen solution,
providing a thrombin solution,
providing a separation medium,
providing a fibrin polymerization device to which said fibrinogen solution, said thrombin solution and said separation medium can be applied and wherein flow of said solutions and medium can be conducted in appropriate ducts for said solutions and medium,
applying to said fibrin polymerization device said fibrinogen solution and said thrombin solution,
transporting said fibrinogen solution and said thrombin solution in ducts of said fibrin polymerization device and contacting said fibrinogen solution with said thrombin solution in the course of said transportation so as to obtain a homogeneous mixture of fibrinogen and thrombin and to allow the polymerization of fibrin,
transporting said mixture in a duct of said fibrin polymerization device in a continuous flow,
applying said separation medium to said fibrin polymerization device, transporting said separation medium in a duct of said fibrin polymerization device and interrupting said continuous flow of said mixture with said separation medium so as to obtain consecutive volumes of said mixture and volumes of said separation medium and wherein said mixture is polymerizing or already polymerized,
further transporting said consecutive volumes of said polymerizing or polymerized mixture and volumes of said separation medium in a duct of said fibrin polymerization device wherein said polymerizing or polymerized mixture optionally further polymerizes to obtain a discontinuous fibrin product, and
removing said discontinuous fibrin product from said fibrin polymerization device.
   12. Method according to embodiment 11, wherein said fibrin polymerization device comprises at least one pressuring device for transporting the solutions and medium.
   13. Method according to embodiment 11 or 12, wherein said pressuring device is a pump or a plunger.
   14. Method according to any one of embodiments 11 to 13, wherein said polymerization device comprises containers for said fibrinogen solution, said thrombin solution and said separation medium.
   15. Method according to any one of embodiments 11 to 14, wherein said polymerization device comprises a mixing device for said fibrinogen and said thrombin solution, said mixing device is preferably selected from the group consisting of a Y-shaped connector, a filter material, a three-dimensional lattice or matrix material.
   16. Method according to embodiment 15 wherein said mixing device is connected with said container for the fibrinogen solution and said container for said thrombin solution by ducts wherein said solutions can be transported from said container to said mixing device.
   17. Method according to any one of embodiments 11 to 16, wherein said ducts are made of a material selected from the group consisting of Polyethylene (PE), High Density Polyethylene (HDPE), Polypropylene (PP), Ultra High Molecular Weight Polyethylene (UHMWPE), Nylon, Polytetra Fluoro Ethylene (PTFE), PVdF, Polyester, Cyclic Olefin Copolymer (COC), Thermoplastic Elastomers (TPF) including EVA, Polyethyl Ether Ketone (PEEK), glass, ceramic, metal, synthetic and natural biodegradable biopolymers, hydro-biodegradable plastics (HBP) and oxo-biodegradable plastics (OBP), PHA (polyhydroxyalkanoates), PHBV (polyhydroxybutyrate-valerate), PLA (polylactic acid), PGA (polygycolic acid), PCL (polycaprolactone), PVA (polyvinyl alcohol), PET (polyethylene terephthalate), Polydimethylsiloxane (PDMS) or silicone rubber.
   18. Method according to any one of embodiments 11 to 17, wherein said separation medium is selected from the group consisting of air, N₂, He, H₂, O₂, Ne, Ar, Kr, Xe, NO, NO₂, CO₂, N₂O, mixtures of such gases, H₂O, an aqueous solution, an organic solvent, media culture for growing cells, medical anaesthesia gases, such as entonox, nitronox or such gases mixed with air; fluorinated ether anaesthetics, such as sevoflurane, isoflurane, enflurane and desfurane; liquids having a higher density than the fibrin segment; insoluble liquids that can be supplemented with an active ingredient.
   19. Method according to any one of embodiments 11 to 18, wherein said fibrinogen solution and/or said thrombin solution further contains a pharmaceutically active additive.
   20. Method according to any one of embodiments 11 to 19, wherein said discontinuous fibrin product is interconnected by polymerized fibrin material.
   21. Method according to any one of embodiments 11 to 19, wherein said discontinuous fibrin product consists of separated volumes of polymer material corresponding to said consecutive volumes of said polymerized mixture.
   22. Method according to any one of embodiments 11 to 21, wherein said duct transporting said polymerizing mixture of fibrinogen and thrombin and said duct transporting said separation medium are connected by a T- or Y-shaped connector.
   23. Method according to any one of embodiments 11 to 22, wherein said ducts and/or connectors have an internal diameter of 0.2 to 5 mm, preferably from 0.6 to 2 mm, especially of 1.2 to 1.6 mm.
   24. Method according to any one of embodiments 11 to 23, wherein said duct wherein said consecutive volumes of said polymerizing or polymerized mixture and volumes of said separation medium are transported in said fibrin polymerization device contains withdrawal means for said separation medium to withdraw said separation medium.
   25. Method according to embodiment 24, wherein said withdrawal means for said separation medium are holes or semipermeable surfaces in said duct or absorption devices for said separation medium in said duct.
   26. Method according to any one of embodiments 11 to 25, wherein said method is conducted in a segmented flow analysis (SFA) format or in a flow injection analysis (FIA) format.
   27. Method according to any one of embodiments 11 to 26, wherein said ducts have an individual length of 1 mm to 10 m, preferably from 0.5 cm to 3 m, especially from 1 to 50 cm.
   28. Method according to any one of embodiments 11 to 27, wherein said volume of said polymerizing or polymerized mixture is from 0.5 to 20 μl, preferably from 1′ to 5 μl.
   29. Method according to any one of embodiments 11 to 28, wherein said transporting is performed at a flow rate of 0.05 to 50 ml/min, preferably of 0.5 to 20 ml/min, especially of 1 to 10 ml/min.

30. Method according to any one of embodiments 11 to 29, wherein said removing of said discontinuous fibrin product from said fibrin polymerization device includes removing the duct wherein said fibrin product is present.

31. Method according to any one of embodiments 11 to 30, wherein said fibrin polymerization device comprises heating and/or cooling means for heating and/or cooling at least parts of the fibrin polymerization device, especially ducts or containers.

32. Method according to any one of embodiments 11 to 31, wherein said fibrin product is lyophilized after removing from said fibrin polymerization device,

33. Fibrin polymer obtainable by a method according to any one of embodiments 11 to 32.

34. Fibrin polymer obtainable by a method according to embodiment 20.

35. Fibrin polymer obtainable by a method according to embodiment 24 or 25.

36. Fibrin polymer obtainable by a method according to embodiment 30.

37. Fibrin polymer according to any one of embodiments 33 to 36 wherein said fibrin polymer is present in lyophilized form.

38. Fibrin polymer according to any one of embodiments 33 to 37 wherein said fibrin polymer has been treated by virus inactivation treatments.

39. Fibrin polymer according to any one of embodiments 33 to 38 wherein said fibrin polymer is provided in a sterile container.

40. Fibrin polymerization device for the production of a fibrin product comprising:
an inlet for a fibrinogen solution,
an inlet for a thrombin solution,
an inlet for a separation medium,
ducts for conducting flow and transport of said solutions and medium, especially means for mixing the solutions and interrupting the continuous flow of said mixture with said separation medium.
   41. Fibrin polymerization device according to embodiment 40 further comprising at least one pressuring device for transporting said solutions and medium.
   42. Fibrin polymerization device according to embodiment 40 or 41, wherein said pressuring device is a pump or a plunger.
   43. Fibrin polymerization device according to any one of embodiments 40 to 42, wherein said polymerization device comprises containers for said fibrinogen solution, said thrombin solution and said separation medium.
   44. Fibrin polymerization device according to any one of embodiments 40 to 43, wherein said polymerization device comprises a mixing device for said fibrinogen and said thrombin solution.
   45. Fibrin polymerization device according to claim 44 wherein said mixing device is selected from the group consisting of a Y-shaped connector, a filter material, a three-dimensional lattice or matrix material.
   46. Fibrin polymerization device according to embodiment 44 or 45 wherein said mixing device is connected with said container for the fibrinogen solution and said container for said thrombin solution by ducts wherein said solutions can be transported from said container to said mixing device.
   47. Fibrin polymerization device according to any one of embodiments 40 to 46, wherein said ducts are made of a material selected from the group consisting of Polyethylene (PE), High Density Polyethylene (HDPE), Polypropylene (PP), Ultra High Molecular Weight Polyethylene (UHMWPE), Nylon, Polytetra Fluoro Ethylene (PTFE), PVdF, Polyester, Cyclic Olefin Copolymer (COC), Thermoplastic Elastomers (TPE) including EVA, Polyethyl Ether Ketone (PEEK), glass, ceramic, metal, synthetic and natural biodegradable biopolymers, hydro-biodegradable plastics (HBP) and oxo-biodegradable plastics (OBP), PHA (polyhydroxyalkanoates), PHBV (polyhydroxybutyrate-valerate), PLA (polylactic acid), PGA (polygycolic acid), PCL (polycaprolactone), PVA (polyvinyl alcohol), PET (polyethylene terephthalate), Polydimethylsiloxane (PDMS) or silicone rubber.
   48. Fibrin polymerization device according to any one of embodiments 40 to 47, wherein said duct transporting said polymerizing mixture of fibrinogen and thrombin and said duct transporting said separation medium are connected by a T- or Y-shaped connector.
   49. Fibrin polymerization device according to any one of embodiments 40 to 48, wherein said ducts and/or connectors have an internal diameter of 0.2 to 5 mm, preferably from 0.6 to 2 mm, especially of 1.2 to 1.6 mm.
   50. Fibrin polymerization device according to any one of embodiments 40 to 49, wherein at least one duct in said fibrin polymerization device contains withdrawal means for the separation medium to withdraw said separation medium.
   51. Fibrin polymerization device according to embodiment 50, wherein said withdrawal means for said separation medium are holes or semipermeable surfaces in said duct or absorption devices for said separation medium in said duct.
   52. Fibrin polymerization device according to any one of embodiments 40 to 51, wherein said ducts have an individual length of 1 mm to 10 m, preferably from 0.5 cm to 3 m, especially from 1 to 50 cm.
   53. Fibrin polymerization device according to any one of embodiments 40 to 52, wherein said volume of said polymerizing or polymerized mixture is from 0.5 to 20 μl, preferably from 1 to 5 μl.
   54. Fibrin polymerization device according to any one of embodiments 40 to 53, wherein said transporting is performed at a flow rate of 0.05 to 50 ml/min, preferably of 0.5 to 20 ml/min, especially of 1 to 10 ml/min.
   55. A kit for assembling a polymerization device according to any one of embodiments 8 to 10 and 40 to 54, comprising ducts, preferably ducts with two or more different inner diameters, at least one polymer mixture inlet, at least one separation medium inlet and at least one flow device.
   56. Kit according to embodiment 55, wherein it additionally comprises at least one polymerization mixture preparation device, at least one duct with holes and/or flanges, polymerization mixture components, preferably fibrinogen, thrombin, collagen, alginate, chitosan, at least one metal ion preparation, at least one photoactivator or mixtures thereof, provided that said mixture does not already constitute a polymerization mixture.

1. Method for the production of a polymerized product comprising the following steps:
providing a polymerization device to which a polymerization mixture and a separation medium can be applied and wherein flow of said mixture and medium can be conducted in appropriate ducts for said mixture and medium,
transporting said polymerization mixture in a duct of said polymerization device thereby allowing the polymerization reaction,
transporting said mixture in a duct of said polymerization device in a continuous flow,
interrupting said continuous flow of said mixture with said separation medium so as to obtain consecutive volumes of said mixture and volumes of said separation medium,
further transporting said consecutive volumes of said mixture and volumes of said separation medium in a duct of said polymerization device wherein said mixture further polymerizes to obtain a discontinuous polymerized product, and
removing said discontinuous polymerized product from said polymerization device.

2. Method according to claim 1, wherein said polymerization mixture is selected from a mixture of fibrinogen and thrombin, a mixture of gelatine and thrombin, a mixture of polysaccharide, especially alginate, and calcium, a mixture of polysaccharide and isocyanate, a mixture of poly(vinyl alcohol)-alginate and calcium, a mixture of albumin and aldehyde, a mixture of chitosan and glutaric dialdehyde, a mixture of chitosan and glycerol-phosphate disodium salt, a mixture of collagen and glutaraldehyde, a mixture of gelatin and glutaraldehyde, a mixture of polyethyleneglycol and amino acid with reactive end groups, a mixture of alginate—polyethyleneglycol diamines and carbodiimide.

3. Method according to claim 1, wherein said polymerization device comprises at least one pressuring device for transporting mixture and medium, said pressuring device is preferably a pump or a plunger.

4. Method according to claim 1, wherein said polymerization device comprises a mixing device for said components so as to obtain said polymerization mixture.

5. Polymerization device suitable for carrying out the method according to claim 1.

6. Method for the production of a fibrin product comprising the following steps:
providing a fibrinogen solution,
providing a thrombin solution,
providing a separation medium,
providing a fibrin polymerization device to which said fibrinogen solution, said thrombin solution and said separation medium can be applied and wherein flow of said solutions and medium can be conducted in appropriate ducts for said solutions and medium,
applying to said fibrin polymerization device said fibrinogen solution and said thrombin solution,
transporting said fibrinogen solution and said thrombin solution in ducts of said fibrin polymerization device and contacting said fibrinogen solution with said thrombin solution in the course of said transportation so as to obtain a homogeneous mixture of fibrinogen and thrombin and to allow the polymerization of fibrin,
transporting said mixture in a duct of said fibrin polymerization device in a continuous flow,
applying said separation medium to said fibrin polymerization device, transporting said separation medium in a duct of said fibrin polymerization device and interrupting said continuous flow of said mixture with said separation medium so as to obtain consecutive volumes of said mixture and volumes of said separation medium and wherein said mixture is polymerizing or already polymerized,
further transporting said consecutive volumes of said polymerizing or polymerized mixture and volumes of said separation medium in a duct of said fibrin polymerization device wherein said polymerizing or polymerized mixture optionally further polymerizes to obtain a discontinuous fibrin product, and
removing said discontinuous fibrin product from said fibrin polymerization device.

7. Method according to claim 6, wherein said fibrin polymerization device comprises at least one pressuring device for transporting the solutions and medium, preferably wherein said pressuring device is a pump or a plunger; and/or wherein said polymerization device comprises a mixing device for said fibrinogen and said thrombin solution, said mixing device is preferably selected from the group consisting of a Y-shaped connector, a filter material, a three-dimensional lattice or matrix material.

8. Method according to claim 6, wherein said discontinuous fibrin product is interconnected by polymerized fibrin material.

9. Method according to claim 6, wherein said duct wherein said consecutive volumes of said polymerizing or polymerized mixture and volumes of said separation medium are transported in said fibrin polymerization device contains withdrawal means for said separation medium to withdraw said separation medium.

10. Method according to claim 9, wherein said withdrawal means for said separation medium are holes or semipermeable surfaces in said duct or absorption devices for said separation medium in said duct.

11. Fibrin polymer obtainable by a method according to claim 6.

12. Fibrin polymer obtainable by a method according to claim 8.

13. Fibrin polymer obtainable by a method according to claim 9.

14. Fibrin polymerization device for the production of a fibrin product comprising:
an inlet for a fibrinogen solution,
an inlet for a thrombin solution,
an inlet for a separation medium,
ducts for conducting flow and transport of said solutions and medium, especially means for mixing the solutions and interrupting the continuous flow of said mixture with said separation medium.

15. A kit for assembling a polymerization device according to claim 5, comprising ducts, preferably ducts with two or more different inner diameters, at least one polymer mixture inlet, at least one separation medium inlet and at least one flow device.
